SEQUENCE LISTING (2 sequences)

SEQ ID NO: 1 - protein, 10 aa, Artificial Sequence
  Synthetic Peptide
KKAHWGFTLD

SEQ ID NO: 2 - protein, 10 aa, Artificial Sequence
  Synthetic Peptide
CTTHWGFTLC

(19) United States
(12) Patent Application Publication
(10) Pub. No.: US 2014/0178293 A1
(43) Pub. Date: Jun. 26, 2014

(54) METHODS FOR IMAGING BONE PRECURSOR CELLS USING DUAL-LABELED IMAGING AGENTS TO DETECT MMP-9 POSITIVE CELLS

(71) Applicants: Elizabeth A. Davis, Missouri City, TX (US); Alan R. Davis, Missouri City, TX (US); Eva M. Sevick-Muraca, Montgomery, TX (US); Sunkuk Kwon, Pearland, TX (US)

(72) Inventors: Elizabeth A. Davis, Missouri City, TX (US); Alan R. Davis, Missouri City, TX (US); Eva M. Sevick-Muraca, Montgomery, TX (US); Sunkuk Kwon, Pearland, TX (US)

(73) Assignee: BAYLOR COLLEGE OF MEDICINE, Houston, TX (US)

(21) Appl. No.: 14/115,990

(22) PCT Filed: May 7, 2012
(86) PCT No.: PCT/US12/36749

§ 371 Date: Nov. 6, 2013

Related U.S. Application Data
(60) Provisional application No. 61/483,600, filed on May 6, 2011.

(51) Int. Cl. A61K 49/00

(52) U.S. Cl. CPC A61K 49/0004
USPC 424/1.11; 424/9.1; 424/9.6

(57) ABSTRACT

The present invention includes embodiments for methods and compositions that identify the presence of or risk for developing heterotopic ossification, particularly prior to mineralization of the bone. In particular embodiments, MMP-9 and/or MMP-2 agents comprising dual imaging moieties are used to identify patterns of MMP-9 and/or MMP-2 localization, respectively, that is then predictive of heterotopic ossification.

### CROSS-REFERENCE TO RELATED APPLICATIONS

This Application claims the benefit of U.S. Provisional Application 61/483,600, filed on May 6, 2011, which is incorporated by reference herein in its entirety.

### STATEMENT REGARDING FEDERALLY SPONSORED RESEARCH OR DEVELOPMENT

The invention was made with government support under R01EB005173 awarded by National Institute of Biomedical Imaging and Bioengineering and under W81XWH-07-1-0214 awarded by the Department of Defense and W911NF-09-1-0040 awarded by Defense Advanced Research Projects Agency. The government has certain rights in the invention.

### TECHNICAL FIELD

The present invention includes at least the fields of cell biology, molecular biology, imaging, diagnostics, and medicine.

### BACKGROUND OF THE INVENTION

Multimodality imaging provides complementary functional and anatomical information for diagnosis, treatment planning, and therapeutic monitoring. Clinical hybrid systems that combine functional imaging modalities such as Positron Emission Tomography (PET) and Single-Photon Emission Computed Tomography (SPECT) with Computed Tomography (CT) offer the ability to obtain molecular imaging data that can be co-registered with anatomical imaging, playing substantial roles in patient care. The development of a single agent capable of carrying dual-contrast for both nuclear and CT or MRI imaging modalities proves challenging due to the inherent differences in measurement sensitivities between nuclear and all other conventional imaging modalities. While the pico- to femto-molar sensitivity of nuclear imaging permits the use of microdosing and minimizes potential pharmacologic effects and toxicity of a radiotracer, MR and CT contrast agents currently require millimolar tissue concentrations for acquisition (Culver et al., 2008).

Near-infrared fluorescence (NIRF) optical imaging is an emerging imaging modality that promises comparable sensitivity to nuclear imaging (Houston et al., 2005; Sevick-Muraca and Rasmussen, 2008). Because of the comparable sensitivity, dual optical/nuclear labeling of molecularly-targeted imaging agents can provide specific advantages. Foremost, if an imaging agent can be dual-labeled with a radionuclide and a NIR excitable fluorophore, then a single imaging agent can be used for non-invasive imaging of diseased tissues (via PET and possibly NIRF imaging) as well as intraoperative guidance for accurate surgical removal of corresponding tissues and tissue margins (via NIR fluorescence imaging). Second, because the NIR signal does not have a physical half-live, it can facilitate the validation of agent targeting capabilities long after physical decay of radiotracer. Lastly, if NIR fluorescent agents are to be used in molecular imaging, dual-labeling provides a strategy for comparative assessment against the conventional nuclear imaging modalities.

To date, the only NIR fluorophore that has been employed in human NIRF imaging studies is indocyanine green (ICG) (Marshall et al., 2010). However, dyes that possess better optical properties (i.e. increased fluorescent yield, preferable hydrophilicity, and enhanced stability) and can be subjected to reaction in organic, aqueous and solid phase chemistries with reduced risk of physical degradation or loss of fluorescence following radiolabeling are of great interest. IRDye 800CW is a NIR dye that is functionalized with either an N-hydroxysuccinimide or maleimide reactive group, allowing it to be attached to a number of biomolecules. Owing to its NIR excitation which abrogates tissue autofluorescence as a complicating background signal (Adams et al., 2007) as well as to its unprecedented stability, IRDye 800CW has been used in a number of preclinical studies (Sampath et al., 2008; Sampath et al., 2007; Chen et al., 2009; Liu et al., 2010; Wang et al., 2004; Tanaka et al., 2008; Cao et al., 2010). While the imaging sensitivity to IRDye 800CW and other NIR fluorophores ultimately depends upon instrumentation design (Sevick-Muraca et al., 2008), efficiency of dual-labeled agents also depends upon the stability and efficient fluorescent yield of the NIR fluorophore following conjugation and radiolabeling.

Different nuclear/optical dual-labeling strategies have been reported for antibody (Sampath et al., 2008; Sampath et al., 2007; Ogaa et al., 2009; Sampath et al., 2010) and peptide-based agents (Becker et al., 2001; Achilefu et al., 2002; Li et al., 2006; Edwards et al., 2008; Kimura et al., 2010). Peptides are ideal molecules for dual-labeling since they can be synthesized by solid-phase peptide synthesis and have a clearly defined structure to which site-specific conjugations can be performed. Also, peptides clear rapidly from circulation and provide high target-to-background ratios at early time points. Rapid clearance is significant as it allows for the use of shorter-lived radionuclides, such as Gallium-68 (⁶⁸Ga, t_{1/2}=68 min), which can be used for PET imaging and limits the overall radiation dose a subject receives. ⁶⁸Ga is a positron-emitting radionuclide that is produced from commercially-available generators. Since the generator is housed locally and can be eluted multiple times per day, it allows for rapid method development and clinical deployment of new radiotracers. ⁶⁸Ga is formed as the decay product of the long-lived parent radionuclide Germanium-68 (⁶⁸Ge, t_{1/2}=270 d), thus allowing routine use of the ⁶⁸Ga-generator for nearly one year.

In embodiments of the present invention, the use of a ⁶⁸Ga/IRDye 800CW dual-labeling strategy for a peptide that targets the gelatinases, matrix metalloproteinases-2 and -9 (MMP-2/-9) was characterized. MMPs are a family of enzymes that participate in extracellular matrix (ECM) degradation. Altered MMP expression has been reported in physiological conditions including rheumatoid arthritis, atherosclerosis, heart failure, pulmonary emphysema, and tumor growth and metastasis, and bone formation (Chang et al., 2008; Lancelot et al., 2008; Muroski et al., 2008; Boschetto et al., 2006; Deryugina and Quigley, 2006; Manduca et al., 2009). A model of heterotopic ossification (HO) has been described using bone morphogenic protein (BMP) signaling which activates MMP9 and contributes to the new bone formation Rodenberg et al., 2010). While there are several strategies for targeting the entire family of MMPs, Koivunen et al. originally described several peptide sequences containing the HWGF motif that showed excellent inhibition of the gelatinases, MMP-2 and MMP-9 (Koivunen et al., 1999). The CTTHWGFTLC (CTT; SEQ ID NO:2) peptide showed the best inhibitory properties and led to its functionalization by others to generate different imaging probes with Iodine-125 (¹²⁵1), Indium-111 (¹¹¹In) and Copper-64 (⁶⁴Cu) labels (Kuhnast et al., 2004; Sprague et al., 2006; Hanaoka et al., 2007). In an attempt to generate a variant of the CTT peptide for fluorescence imaging, Wang et al. modified the N-terminus in order to attach a red-excitable fluorophore, Cy5.5, and to improve the in vivo stability of the peptide (Wang et al., 2009). This agent showed specific tumor uptake and served as the basis for the NIR/PET dual-labeled compound described herein.

The present invention encompasses methods and compositions that provide solutions to a long-felt need in the art of informative imaging of heterotopic ossification.

### BRIEF SUMMARY OF THE INVENTION

In some embodiments of the invention, there are methods and compositions related to agents for use in the identification of or prediction of heterotopic bone formation (which may also be referred to as heterotopic ossification) and/or prevention thereof. In particular aspects, one or more detectable agents are utilized to identify at least one marker of heterotopic bone formation. In specific aspects, the identification of the marker(s) occurs prior to mineralization of the bone.

In some aspects of the invention, there is a system to identify heterotopic bone growth or the prediction thereof. In specific cases, an individual is in need of identification of heterotopic bone or the risk for developing heterotopic bone growth. An individual may be in need of identification of heterotopic bone or at risk for developing heterotopic bone growth if they have been subjected to an event that deleteriously impacts one or more bones, and in specific embodiments the methods and compositions of the invention are employed following the event but prior to heterotopic bone growth or prior to mineralization of heterotopic bone. In specific cases, the event that deleteriously impacts one or more bones is trauma, for example, and the trauma may be singular (such as an injury) or the accumulation of events, such as repetitive physical stress of bones (such as a bone spur). In some embodiments, an individual having a joint replacement (including hip, knee, jaw, elbow, and shoulder) is locally provided one or more agents of the invention during and/or after the replacement. Particular embodiments of the invention encompass use of the methods and compositions no later than 24 hours to two years following an event that may result in heterotopic bone growth, such as trauma.

An individual at risk for developing heterotopic ossification may have one or more of the following risk factors: male gender; has active ankylosing spondylitis; has diffuse Idiopathic Skeletal Hyperostosis; has post traumatic arthritis; has heterotrophic osteoarthritis; has previous heterotopic ossification; had previous hip fusion; has Paget's disease; has Parkinson's disease has excessive osteophytosis or enthesiopathic radiographic changes on AP of pelvis; has traumatic brain injury and/or spinal cord injury and/or stroke; has had hip surgery or other joint surgery; has burns; has long period of immobility; has a joint infection; has trauma to muscle or soft tissue.

In at least certain aspects, methods and compositions of the invention are utilized as an alternative to or in addition to surgical removal of heterotopic bone.

In some embodiments of the invention, there is employment of one or more agents for diagnosis and therapy of heterotopic ossification.

In particular embodiments, one or more agents are employed that target an early marker of heterotopic bone growth, such as MMP-9 and/or MMP-2. In specific cases, the agent(s) target a marker in its activated form.

The compositions and methods of the invention may be employed for temporal and/or spatial boundaries of present or future bone heterotopic growth.

In some embodiments of the invention, there is employment of one or more agents for diagnosis and therapy of abnormal bone cell growth. In specific embodiments, the agent(s) is utilized in methods following a direct or indirect BMP-induced induction of MMP-9 and/or MMP-2 expression and, in specific embodiments, prior to mineralization. In specific aspects, the methods and compositions are not employed subsequent to the onset of mineralization of bone.

In some embodiments of the invention, there is a detectable agent that effectively targets an early marker of heterotopic bone growth, and in specific embodiments the detectable agent is modified with one or more imaging moieties. In specific embodiments, when there are two imaging moieties they may be detectable by different means. In certain aspects, the moieties that are distinguished by different imaging methods may be optical, radioactive, fluorescent, by color, and so forth.

In specific embodiments of the invention, one or more agent(s) for detection of heterotopic bone formation are useful following expression of the marker to which the agent is targeted. In particular aspects, the agent is useful to determine a region wherein heterotopic bone formation is occurring or will occur or is likely to occur. In certain embodiments, there is a correlation between the imaged localization of the agent and the formation of heterotopic bone in a tissue. In specific cases, there is a substantially identical pattern of detectable localization of the agent and the present or eventual formation of HO bone growth.

In particular embodiments of the invention, following utilization of methods and compositions of the invention that result in detection of present or potential heterotopic bone growth, there is employment of one or more therapeutic agents to prevent or reduce heterotopic bone growth, including localized delivery of the one or more therapeutic agents. Any therapeutic agent may be used, although in specific embodiments the agent prevents heterotopic bone growth following MMP-9 and/or MMP-2 expression. Exemplary therapeutic agents include one or a combination of bisphosphonates (see, for example, U.S. Pat. No. 5,196,409), ehtylhydroxydiphosphonets (EHDP); anti-inflammatory drug, such as a prostaglandin synthase inhibitor; radiation therapy; ibuprofen; aspirin, one or more methods or compositions of U.S. Pat. No. 7,378,395, sodium chromolyate; antagonists of substance P, antagonists of TRVP1, antagonists of RAR-alpha, and/or inhibitors of MMP9. In at least specific cases, the methods and compositions of the invention are informative as to when and/or where therapeutic agents may be used for prevention or reduction of heterotopic bone growth formation.

The methods and compositions of the invention may be employed for heterotopic bone growth of any kind, including within any tissue. In specific cases, the invention is useful for heterotopic bone growth in the muscle, blood vessels (including of the heart or brain, for example), aortic valve, ligaments, and/or tendons.

In particular embodiments, the methods and compositions are utilized in an individual that has or is at risk for developing atherosclerosis, aortic stenosis, calcific aortic valve disease, aneurysm, spinal cord injury, joint replacement, or amputation, and so forth. In some embodiments of the invention, one or more agents of the invention are used upon aortic valve replacement, and in specific cases the agent(s) are delivered locally at the valve replacement site. In embodiments wherein the invention is employed for aneurysm risk, it is known that a lot of aneurysms or rupture of the vessels occur because the plaque is hard (ossified) and therefore will pull away from the wall with routine movement, leading to rupture and death; scans pursuant to the inventive embodiments would aid in providing information of “risk” of vessel wall rupture from a plaque.

In some cases, the methods and compositions of the invention may be utilized in models for determining effectiveness of a drug for heterotopic formation. For example, a drug being tested for therapeutic effectiveness against heterotopic formation may be provided to a test system, such as a rodent model, to identify localization of MMP-9 and/or MMP-2 or another marker of heterotopic bone formation or risk of developing heterotopic bone formation. That is, a model of heterotopic formation may be subjected to the test drug locally following delivery of the agent(s) of the invention for imaging of MMP-9 and/or MMP-2, and the development of heterotopic formation is subsequently determined. When the test drug is effective in this system to prevent or reduce heterotopic formation, the test drug may be employed therapeutically in an effective amount of a human.

In at least certain cases, one or more MMP-9 and/or MMP-2 targeting agents are utilized at a site suspected of or known to be susceptible to heterotopic formation.

The targeting agents of the invention, in at least some embodiments, utilize optical and PET imaging techniques for detectability; in specific cases, micro amounts of the agents are employed in methods of the invention.

In some cases of the invention, a targeting agent is used for diagnostic and therapeutic purposes related to heterotopic bone growth. For example, a MMP-9 and/or MMP-2 targeting agent may include dual imaging moieties such that localization of the agent is indicative of present or potential heterotopic bone growth, and the MMP-9 and/or MMP-2 targeting agent itself is useful to reduce or prevent the present or subsequent heterotopic bone growth.

Certain embodiments of the invention are employed for the monitoring of effectiveness of a treatment for heterotopic bone growth. For example, one or more MMP-9 and/or MMP-2 targeting agents may be delivered to a site suspected of or at risk for developing heterotopic bone growth, a treatment for heterotopic bone growth is delivered to the site, and a subsequent delivery of one or more MMP-9 and/or MMP-2 targeting agents are delivered to the site. When there is a reduction in the quantity and/or localization of imaged MMP-9 and/or MMP-2 targeting agents following the treatment, the treatment is considered effective. When the intensity and/or localization of the imaged MMP-9 targeting agents is not reduced when compared to the pattern prior to treatment, the treatment is not considered effective.

Some embodiments of the invention include additional methods to assay bone presence, such as a bone scan or x-ray or early blood tests for bone formation, for example.

In some embodiments of the invention, there is characterization of chemical, radiochemical and optical properties of a dual-labeled MMP-9 and/or MMP-2 targeting peptide.

Some embodiments of the invention concern MMP-9 and/or MMP-2 as a biomarker of heterotopic ossification.

In some embodiments, there is a method of identifying a site of heterotopic bone formation in an individual or identifying a site at risk of developing heterotopic bone formation in an individual, comprising the step of providing to a localized site in the individual a MMP-9 and/or MMP-2 targeting agent, said agent comprising a first and a second imaging moiety. In particular embodiments, the localized site is muscle, a vessel, a joint, aortic valve, tendons, or ligaments, and the localized site is selected from the group consisting of: a) a trauma site; b) a site subjected to repetitive motion; c) a site at risk for aneurysm; and d) joint arthoplasty.

In some embodiments of the invention, an individual has or is at risk of having traumatic brain injury, atherosclerosis, aortic stenosis, calcific aortic valve disease, spinal cord injury, joint replacement, or amputation. The individual may have one or more of the risk factors selected from the group consisting of male gender; has active ankylosing spondylitis; has diffuse Idiopathic Skeletal Hyperostosis; has post traumatic arthritis; has heterotrophic osteoarthritis; has previous heterotopic ossification; had previous hip fusion; has Paget's disease; has Parkinson's disease has excessive osteophytosis or enthesiopathic radiographic changes on AP of pelvis; has traumatic brain injury and/or spinal cord injury and/or stroke; has had hip surgery or other joint surgery; has burns; has long period of immobility; has a joint infection; has trauma to muscle or soft tissue; and a combination thereof.

In some embodiments of the invention there is an agent that is a peptide, small molecular, polypeptide, antibody, nucleic acid, or combination thereof.

In certain aspects of the invention, the first and second imaging moieties are selected from the group consisting of an optical imaging moiety, a fluorescent imaging moiety, and a nuclear imaging moiety. In specific embodiments, a first imaging moiety is a radionuclide and a second imaging moiety is a NIR fluorophore.

Any agent of the invention may be delivered locally, such as delivered by injection.

Some methods of the invention further comprise the step of providing to the individual a therapeutically effective amount of one or more agents that treat heterotopic bone growth. In particular cases, a MMP-9 and/or MMP-2 targeting agent is the agent that treats heterotopic bone growth. In some cases, the one or more agents that treat heterotopic bone growth is physical therapy, bisphosphonate drug; nonsteroidal anti-inflammatory drugs (NSAIDs); radiation therapy; and/or surgery, TRVP1 antagonists, RAR alpha antagonists, substance P antagonists, MMP9 inhibitors, or a combination thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 provides chemical structures of HWGF peptide, DOTA-derivatized peptide (M₁) and dual-conjugate (M₂).

FIG. 2 shows HPLC traces for M₁, IRDye800CW and M₂. Traces were acquired at 280 nm or with a fluorescence detector.

FIG. 3 demonstrates the effects of increasing peptide amount on radiochemical purity of ⁶⁸Ga-M₂. Data represent mean values (%)±SD.

FIG. 4 provides HPLC chromatograms for ⁶⁸Ga-M₂: UV at 280 nm (a), fluorescent (b), and radiometric (c).

FIG. 5 shows stability studies for ⁶⁸Ga-M₂in PBS, DTPA challenge and serum.

FIG. 6 shows multimodality imaging of mice with fracture putty implants. Human fibroblast cells transduced with AdBMP2 were injected into the right hind limb of NOD/SCID mice. Mice were injected with ⁶⁸Ga-M₂. NIR images (A,C) were acquired on day 4 post-implantation and follow-up CTs (B,D) were taken on day 11. Solid arrows indicate site of new bone formation and agent accumulation. Dashed arrows designate control (empty cassette) injection sites in the contralateral limb.

FIG. 7 provides an exemplary schema wherein the modified HWGF cyclic peptide is conjugated to DOTA on solid support (i) yielding M1, labeled with IRDye800 in solution phase (ii) to form M2, and radiolabeled (iii).

FIG. 8 shows photomicrographs of tissues stained with hematoxylin and eosin (H and E) after intramuscular injection of AdBMP2 or Adempty cassette transduced cells into the mouse hindlimb. Soft tissues were isolated, processed, paraffin embedded and sectioned across the entire limb. Every 5^{th} slide was H and E stained, and images representing the reactive area, which immediately surrounds the injected AdBMP2 (A, C and E) or AdEmpty cassette (B, D, and F) at days 2 (A and B); day 6 (C and D); and day 10 (E and F).

FIG. 9 provides photomicrographs of immunofluorescence staining for MMP-9 (red) neurofilament (green) and von Willibrand factor (VWF; yellow), in tissues following induction of HO by delivery of AdBMP2 or Ad-empty cassette transduced cells. Tissues isolated at daily intervals were serially sectioned, and every 5th slide stained and representative images are shown.

FIG. 10A shows quantification of MMP-9 and MMP-2 RNA in tissues after delivery of AdBMP2 or Adempyt cassette transduced cells. Total RNA was isolated and subject to quantitative Real-Time PCR. Each assay was performed in triplicate with n=8 biological replicates per time point.* denotes statistically significant change as determined by a standard T-test, in the sample from the control; p<0.05 B. Quantification of MMP-9 protein and functional activity by ELISA. Tissues were isolated at daily intervals from animals receiving either AdBMP2 or Adempty cassette transduced cells and protein extracts generated. MMP-9 protein was bound through ELISA, and then substrate added, to quantify MMP-9 functional activity. Standard amounts of active MMP-9 were assayed in order to calculate active protein amount from the activity measurements. Statistically significance was calculated using a standard t-test, with n=8.* denotes statistical significance.

FIG. 11 shows μPET/CT imaging of new bone formation with ⁶⁴Cu-M₂. Panel A shows the fused μPET/CT image at day 4 post-implantation (left), the day 4 μCT alone (center) and the day 11 CT. Panel B shows the fused μPET/CT image from the blocking study.

FIG. 12 shows multimodality imaging of new bone formation with ⁶⁴Cu-M₂. NIR fluorescence (A) and μPET/CT and images (B) were acquired on days 2, 4 and 6 post-implantation. The follow-up μCT scans (C) were performed on day 9, 11 and 13 post-implantation to confirm the presence of HO and correlate with agent uptake observed by molecular imaging.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. Embodiments discussed in the context of methods and/or compositions of the invention may be employed with respect to any other method or composition described herein. Thus, an embodiment pertaining to one method or composition may be applied to other methods and compositions of the invention as well.

The term “heterotopic bone formation” or “heterotopic ossification” as used herein refers to bone growth at an abnormal site, including extraskeletal soft tissues.

The term “mineralization” as used herein refers to osteoid or collagen type 1 matrix that possesses modified hydroxylapatite or bone mineral.

### I. General Embodiments of the Invention

Heterotopic ossification (HO) is a serious disorder that occurs when there is aberrant bone morphogenic protein (BMP) signaling in soft tissues. Currently, there are no methods to detect HO before mineralization occurs. Yet once mineralization occurs, there are no effective treatments to reverse HO. Herein, the inventors used confirmatory ex vivo tissue analyses and in vivo molecular imaging of an established murine animal model of BMP-induced HO to show that MMP-9 can be detected as an early-stage biomarker prior to mineralization. Ex vivo analyses show active MMP-9 protein is significantly elevated within tissues undergoing HO as early as 48 hours after BMP induction, with its expression co-localizing to nerves and vessels. In vivo molecular imaging with a dual-labeled near-infrared fluorescence and BPET agent specific to MMP-2/-9 expression paralleled the ex vivo observations and reflected the site of HO formation as detected from BCT seven days later. The results indicate that the MMP-9 is a biomarker of the early extracellular matrix (ECM) re-organization and is useful as an in vivo diagnostic for detecting HO or conversely for monitoring the success of tissue-engineered bone implants that employ ECM biology for engraftment.

### II. MMP-9 and/or MMP-2 Targeting Agents

The present invention encompasses one or more targeting agents for MMP-9 and/or MMP-2 such that their utilization provides information to predict or identify heterotopic ossification prior to mineralization. The agents target MMP-9 and/or MMP-2, and the skilled artisan recognizes that exemplary protein sequences for them are available in the art, such as at the National Center for Biotechnology Information's GenBank® database, wherein Accession No. P08253 is representative of MMP-2 and Accession No. CAC07541.1 is representative of MMP-9 (both of which are incorporated by reference herein), for example.

The targeting agent is suitable so long as it is capable of identifying the location of MMP-9 and/or MMP-2, such as by indirect or direct binding of the agent to MMP-9 and/or MMP-2, respectively. In certain embodiments, the agent is a peptide, although the agent may be a small molecule, polypeptide, antibody, nucleic acid (including siRNA, shRNA, miRNA, and so forth), or a combination thereof.

The present invention includes MMP-9 and/or MMP-2 targeting agents whose location are detectable by at least one imaging method, although in specific embodiments the agent is detectable by more than one imaging method, including at least two or three methods. In particular cases, the MMP-9 and/or MMP-2 targeting agent has one, two, or more imaging moieties that are covalently or otherwise linked to the agent. An imaging moiety may be linked to an agent by conjugation/The imaging moiety or moieties may be of any kind, although in specific embodiments they are optical, nuclear (radioactive), fluorescent, or colored. The imaging moieties on the agent will allow non-invasive imaging, in particular cases. The imaging moieties will allow intraoperative guidance for accurate surgical removal of corresponding tissue and tissue margins, in at least some cases.

When the MMP-9 and/or MMP-2 targeting agent has two or more imaging moieties, the moieties are attached to the agent sufficiently far apart such that they may be separately detected to identify the same targeting agent molecule. In some embodiments, the agent is or must be modified such that the imaging moieties are sufficiently separated. For example, a linear peptide may need to be modified such that it is cyclic prior to linkage of the first or second imaging moiety.

A. Agent Composition

The MMP-9 and/or MMP-2 targeting agent allows visualization of MMP-9 and/or MMP-2, respectively, to allow one to determine the presence, including specific localization of, heterotopic ossification in a window following expression of MMP-9 and/or MMP-2 but before mineralization of the bone.

In certain embodiments, the agent composition is a peptide, although the agent may be a small molecule, polypeptide, antibody, nucleic acid, or a combination thereof. In specific cases the agent is a circular peptide.

In specific embodiments of the invention, an agent that targets MMP-9 and/or MMP-2 is a peptide comprising a HWGF motif. An exemplary peptide that may be used in the invention is CTTHWGFTLC (SEQ ID NO:2) or KKAHWGFTLD (SEQ ID NO:1), although is some cases the N-terminus or C-terminus is modified. In certain cases the agent is modified to render it stable in vivo, and the skilled artisan is aware of routine methods in the art to achieve this modification. In certain embodiments, a peptide is modified to allow more than one reactive amine group or another modification.

Agents that may be employed as MMP-9 and/or MMP-2 targeting agents may also function as MMP inhibitors, such as ARP 101 ((R)—N—Hydroxy-2-(N-isopropoxybiphenyl-4-ylsulfonamido)-3-methylbutanamide); ARP 100 (2-[((1,1′-Biphenyl)-4-ylsulfonyl)-(1-methylethoxy)amino]-Nhydroxyacetamide); Batimastat ((2R,3S)—N—Hydroxy-N1-[(1S)-2-(methylamino)-2-oxo-1-(phenylmethyl)ethyl]-2-(2-methylpropyl)-3-[(2-thienylthio)methyl]butanediamide); CL-82198 hydrochloride (N-[4-(4-Morpholinyl)butyl]-2-benzofurancarboxamide hydrochloride); Marimastat ((2S,3R)—N4-[(1S)-2,2-Dimethyl-1-[(methylamino) carbonyl]propyl]-N1,2-dihydroxy-3-(2-methylpropyl)butanediamide); ONO-4817 (N—R1S,3S)-1-[(Ethoxymethoxy)methyl]-4-(hydroxyamino)-3-methyl-4-oxobutyl]-4-phenoxybenzamide); PD 166793 (N—R4′-Bromo[1,1′-biphenyl]-4-yl)sulfonyl]-L-valine); Ro 32-3555 ((αR,βR)-β-(Cyclopentylmethyl)-N-hydroxy-γ-oxo-α-[(3,4,4-trimethyl-2,5-dioxo-1-imidazolidinyl)methyl]-1-piperidinebutanamide); UK 370106 (((βR)-β-[[[(1S)-1-[[[(1S)-2-Methoxy-1-phenylethyl]amino]carbonyl]-2,2-dimethylpropyl]amino]carbonyl]-2-methyl-[1,1′-biphenyl]-4-hexanoic acid); WAY 170523 (N-[2-[4-[[[2-[Hydroxyamino)carbonyl]-4-6-dimethylphenyl](phenylmethyl)amino]sulfonyl]phenoxy]ethyl]-2-2-benzofurancarboxamide, all of which may be obtained commercially (AnaSpec, Fremont, Calif.).

The MMP-9 and/or MMP-2 targeting agent may comprise a zinc binding group. They may be hydroxamate based MMP inhibitors, non-hydroxamate based MMP inhibitors (e.g. SB-3CT), novel MMP inhibitors (such as barbiturates), synthetic peptides and pseudopeptides (Hu et al., 2005) (e.g. Regasepin 1) or other inhibitors of MMPs (Paemen et al., 1995) (e.g. REGA-3G12); or RO-28-2653, which belongs to the class of pyrimidine-2,4,6-triones (barbiturates) (Grams et al., 2001).

Vandooren et al. (2011) describe particular MMP-9 inhibitors that may be employed in the invention, including 5-[(2-hydroxy-6-methyl-3-quinolinyl)methylene]-2,4,6(1H,3H,5H)-pyrimidinetrione; N-[4-(6-methyl-1,3-benzothiazol-2-yl)phenyl]tetrahydrothiophene-2-carboxamide; RO-206-0222; N-(4-ethoxy-8-methyl-2-quinazolinyl)guanidine; and N-(2,4-dimethylphenyl)-2-[(2-methyl-1,3-benzothiazol-6-yl)sulfonylamino]acetamide.

B. Radionuclide Moieties

In cases where an imaging moiety is a radionuclide, the radionuclide may be of any kind so long as the half life is of a sufficient duration to allow time to detect the localization of the targeting agent in vivo, yet not so long as to subject the individual to deleterious radiation exposure. In specific embodiments the radionuclide has a half life that is a short-lived radionuclide. Although in specific embodiments the radionuclide is Gallium-68 (⁶⁸Ga), in some cases the radionuclide is Iodine-125 (¹²⁵I), Indium-111 (¹¹¹In), or Copper-64 (⁶⁴Cu).

In particular aspects of the invention, the radionuclide as an imaging agent allows the use of microdosing of the agent, for example in the pico- to femto-molar sensitivity.

In specific aspects of the radionuclide, it is a positron-emitting radionuclide.

In particular embodiments, the radionuclide imaging moiety is measured by positron emission tomography (PET) or near-infrared fluorescence imaging.

C. Near-Infrared (NIR) Moieties

In some embodiments, the targeting agent of the invention employs a NIR moiety to provide high spatial resolution of NIR imaging. The NIR moiety is advantageous at least because it does not have a physical half-life and can be used intra-operatively, allowing light visualization in real time. In particular cases the NIR moiety is a NIR excitable fluorophore. The NIR moiety allows validation of agent targeting capabilities following decay of a radiotracer, for example.

In certain cases the moiety is a near infrared fluorescence moiety. In some cases, the NIR moiety is IRDye 800CW, indocyanine green (ICG), IRDye 680RD, IRDye 680LT, IRDye 750, IRDye 700DXs, IRDye 800RS, or IRDye 650.

### III. Peptides of the Invention

In certain embodiments, the MMP-9 and/or MMP-2 targeting agent concerns compositions comprising at least one proteinaceous molecule, including a peptide of from about 3 to about 100 amino acids, a protein of greater than about 100 amino acids or the full length endogenous sequence translated from a gene.

In certain embodiments the size of the at least one proteinaceous molecule may comprise, but is not limited to, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance that produces no significant untoward effects when applied to, or administered to, a given organism (such as a mammal, including a human, cat, dog, horse, cow, and so forth) according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's GenBank® and GenPept databases. The coding regions for these known genes may be amplified and/or expressed using the techniques disclosed herein or as would be known to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

### IV. Pharmaceutical Preparations

Pharmaceutical compositions of the present invention comprise an effective amount of one or more MMP-9 and/or MMP-2 targeting agents or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one MMP-9 and/or MMP-2 targeting agents or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The MMP-9 and/or MMP-2 targeting agent may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The MMP-9 and/or MMP-2 targeting agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include MMP-9 and/or MMP-2 targeting agent(s), one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the MMP-9 and/or MMP-2 targeting agent may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the MMP-9 and/or MMP-2 targeting agents are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, MMP-9 and/or MMP-2 targeting agents may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound MMP-9 and/or MMP-2 targeting agents may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### V. Kits of the Invention

Any of the compositions described herein may be comprised in a kit. In a non-limiting example, one or more MMP-9 and/or MMP-2 targeting agents may be comprised in a kit in suitable container means. In particular cases, the MMP-9 targeting agent has dual imaging moieties or the kit may comprise one or more imaging moieties to be added to the agent and suitable reagents to do so.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

The components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Experimental Procedures

Reagents

All reagents were purchased from commercial sources and used without further purification. Chelex-100 resin was purchased from Bio-Rad Laboratories (Richmond, Calif.) and used with all aqueous buffers to ensure metal-free conditions. The DOTA (1,4,7,10-tetraazacyclotetradecane-N′,N″,N″′,N″″-tetraacetic acid)-modified cyclic MMP-targeting peptide (lactam 2,10) DOTA-KKAHWGFTLD (M₁) (SEQ ID NO:1) was synthesized by New England Peptide (Gardner, Mass.) according to standard Fmoc-protocols. A commercially-available ⁶⁸Ge/⁶⁸Ga generator was purchased from Eckert & Ziegler (Berlin, Germany). Analytical high-performance liquid chromatography (HPLC) was performed on a Hitachi LaChrom system equipped with a 2.6 μm Kinetex C-18 column (Phenomenex, Torrance, Calif.) with a mobile phase of A=0.1% TFA in H₂O, B=0.1% TFA in CH₃CN; gradient, 0 min=10% B, 10 min=90% B; flow rate, 1 mL/min. Radio-thin-layer chromatography (radio-TLC) was carried out on a AR-2000 scanner (Bioscan, Washington, D.C.) using instant thin-layer chromatography (ITLC) strips and 1:1 methanol/0.1 M ammonium acetate. Molecular weight measurement was carried out by ESI on a Waters UPLC system equipped with a Waters PDA detector and a Waters TQD mass spectrometer.

Conjugation of IRDye 800CW

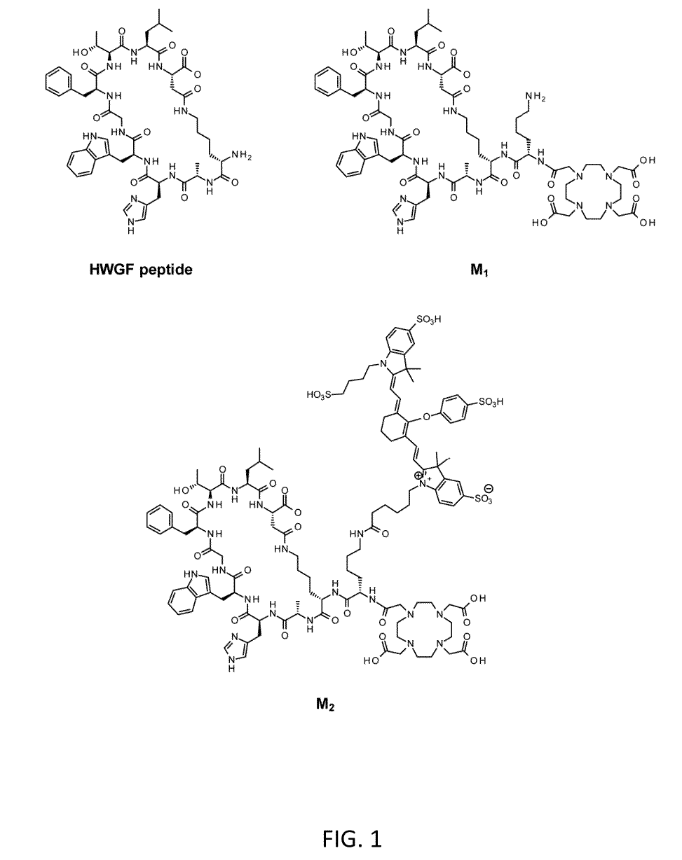

In order to generate a peptide with two reactive amine groups for conjugation to DOTA and IRDye 800CW, the inventors modified the HWGF peptide sequence described by Wang et al. by adding an additional lysine to the N-terminus of the peptide followed by DOTA-conjugation by solid-phase synthesis to yield the cyclic decapeptide M₁ (FIG. 1). Conjugation of IRDye 800CW (LICOR Biosciences, Lincoln, Nebr.) was performed by adding 1.52 mg (1.3 μmol) of dye to a stirred solution of M₁ (2 mg, 1.3 μmol) in 0.1 M sodium phosphate buffer (pH 8.33). The reaction was carried out at 4° C. overnight and the crude mixture was purified with a 2000 MWCO Sartorius Vivaspin spin column (VWR International) or semi-preparative-HPLC to yield M₂.

Radiolabeling

⁶⁸Ga-M₂ was prepared by eluting a 10 mCi ⁶⁸Ge/⁶⁸Ga generator with 0.1 N HCl and collecting the 2 mL peak fraction. The eluted ⁶⁸GaCl₃ was buffered to pH 4 with solid NaOAc. The effects of peptide concentration, buffer concentration, and heating time were tested to determine optimal labeling conditions. Different peptide amounts were added to 0.1 M or 1.25 M NaOAc buffer (pH 4) with reaction volumes ranging from 150-710 μl. Samples were heated at 95° C. for 5-20 min. For pharmacological studies, 1 N NaOH (30 μL) was used to adjust to pH 7. Radiochemical purity was assessed by radio-TLC and confirmed by radio-HPLC.

Synthesis of ^{nat}Ga-M₂

^{nat}Ga-M₂ was synthesized based on conditions developed with ⁶⁸Ga. M₂ (150 μg, 60 nmol) was mixed with an excess of non-radioactive Ga and the reaction was heated at 95° C. for 13 min. The crude mixture was HPLC purified and characterized by mass spectrometry.

Stability Studies

The chemical, radiochemical and optical stability of radioactive and non-radioactive M₂ were examined using a series of in vitro stability studies. M₂ was incubated in PBS and water at room temperature and 4° C. for 14 days, followed by HPLC analysis of peptide and fluorescent stability. To assess radiochemical stability, ⁶⁸Ga-M₂ was added to a solution of PBS or DTPA (500-fold excess), kept at room temperature for 1, 2 and 3 h, and analyzed by radio-HPLC. To evaluate serum stability, 150 μl of ⁶⁸Ga-M₂ was added to 50% mouse serum and incubated at 37° C. An 80 μl aliquot was taken at each of the above-mentioned time points and added to 160 μl ice-cold acetonitrile. The samples were centrifuged at 14,000 rpm for 5 min and the supernatant was collected, filtered and analyzed by radio-HPLC.

Determination of Log P Value

The lipophilicity of ⁶⁸Ga-M₂ was assessed by determination of the water-octanol partition coefficient. 1-Octanol (1 mL) was added to a solution of approximately 25 μCi of ⁶⁸Ga-M₂ in water (1 mL) and the layers were vigorously mixed for 5 min at room temperature. The tubes were centrifuged at 14,000 rpm for 2-3 min. Three samples of 100 μL of each layer were taken in pre-weighed vials, re-weighed, and counted in a μ-counter (Wizard-2, Perkin Elmer). The partition coefficient was determined by calculating the ratio of counts per minute (cpm) in weight (g) of octanol/cpm in weight (g) of water and expressed as log P. At least three independent experiments were performed in triplicate to give the log P as the mean value±standard deviation (SD).

Characterization of Fluorescent Properties

Fluorescence intensity of the fluorophores was determined using the Fluorolog Tau-3 Spectrofluorometer (Horiba Jobin Yvon, Edison, N.J.) with excitation from a xenon arc lamp and absorbance was recorded using the DU-800 Spectrophotometer (Beckman Coulter, Brea Calif.). Fluorescence excitation and emission spectra were obtained at wavelengths of 785 and 830 nm respectively, with an integration time of 0.3 seconds for 1 μM solutions of IRDye 800CW, M₂ and ⁶⁸Ga-M₂ (n=4). Fluorescence measurements for IRDye 800CW and M₂ were performed in aqueous solution under ambient conditions, whereas ⁶⁸Ga-M₂ was prepared as previously described in NaOAc (pH 4) to provide an accurate representation of the ⁶⁸Ga-labeling scheme. Extinction coefficients were determined from the slope of absorbance at 785 nm as a function of the concentration of serial dilutions of each agent. Fluorescence quantum yield was determined by the comparative method of Williams et al. (Williams et al., 1983) using the quantum yield of ICGO (Φ=0.016) at 785/830 nm as a standard.

Animal Model

All animal studies were performed in accordance with the standards of Baylor College of Medicine (Houston, Tex.), Department of Comparative Medicine and The University of Texas Health Science Center (Houston Tex.), Center for Molecular Imaging after review and approval of the protocol by their respective Institutional Animal Care and Use Committee (IACUC) or Animal Welfare Committee (AWC). A murine model of BMP-2 induced HO was used as previously described (Rodenberg et al., 2010). Briefly, human fibroblast (MRCS) cells transduced with either Adempty (control) or AdBMP2 were injected intramuscularly into each hind limb quadriceps muscle of nonobese diabetic/severely compromised immunodeficient (NOD/SCID) mice. Control transduced cells were injected into the contralateral limb. The data showed that elevated RNA protein expression and active MMP-9 content in tissues injected with AdBMP2 transduced cells were maximal 4 days after implantation and were significantly elevated when compared to the contralateral tissues receiving Adempty transduced cells.

NIRF Imaging

Based on the findings from the ex vivo analysis, mice were imaged on day 4 post-implantation with NIRF imaging to assess localization and in vivo fluorescence of M₂ following ⁶⁸Ga-labeling. For all imaging procedures, mice were anesthetized with 1% isoflurane. NIR fluorescence images were acquired 18 hr after intravenous administration of ⁶⁸Ga-M₂ using a custom-built fluorescence imaging systems previously described Houston et al., 2005). Briefly, a field of view was illuminated with 785 nm of light from a laser diode, outfitted with a convex lens and diffuser to create a uniform excitation field. The fluorescence was collected through holographic and interference filters placed before a Nikon camera lens. The images were finally captured by an electron-multiplying charge-coupled device camera (PhotonMax 512; Princeton Instruments, Princeton, N.J.) with 200 to 400 milliseconds of integration time. For acquisition of white-light images, the optical filters were removed, and a low-power lamp illuminated the subject. Image acquisition was accomplished by V++ software (Auckland, New Zealand).

PET/CT Imaging

To visualize the in vivo distribution of the dual-labeled peptide, μPET/CT imaging was performed on day 4 post-implantation using a Siemens Inveon μPET/CT scanner (Siemens Medical, Knoxville, Tenn.) with instrument parameters as previously described (Sampath et al., 2010). The anesthetized mice were injected intravenously with ⁶⁸Ga-M₂ (200 μCi, 6 nmol) and μPET/CT images were acquired 1 h post-injection. To visualize the formation of ectopic bone, CT imaging was performed on days 4 and 11 post-implantation

### Example 2

### Conjugation of IRDye 800CW

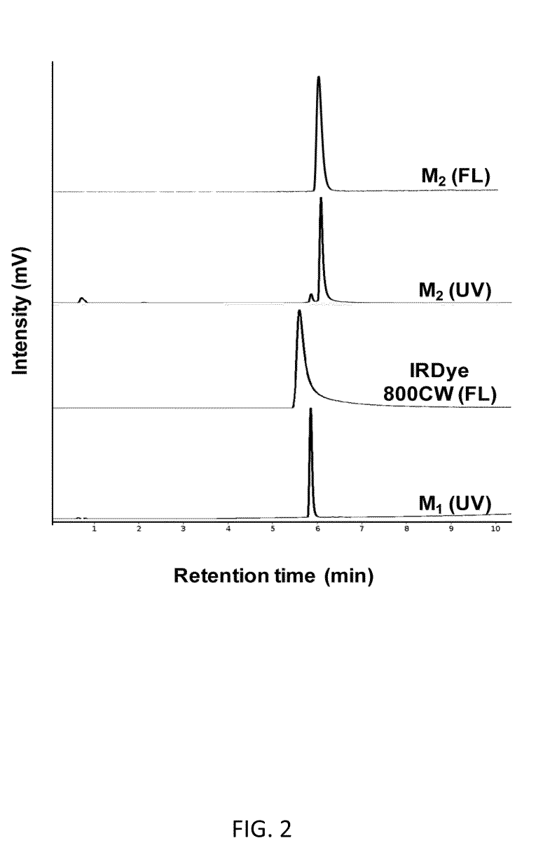

The dual-conjugate M₂ was formed through attachment of IRDye 800CW to M₁. Analysis of the spin column-purified sample by HPLC showed >90% purity with a small amount of unreacted M₁ present in the final product and was used for all subsequent studies. FIG. 2 shows HPLC chromatograms with UV detection at 280 nm and fluorescence detection. Retention times of 5.9 min and 6.1 min were observed for M₁ and M₂ (at 280 nm), respectively, while fluorescence detection of IRDye 800CW had a retention time of 5.6 min. The HPLC data shows a single fluorescent peak for M₂, confirming formation and purity of the dual-conjugate. Analysis by ESI-MS showed that the observed molecular weight (2555.9) was in excellent agreement with the calculated value (2555.92).

### Example 3

### Radiochemistry

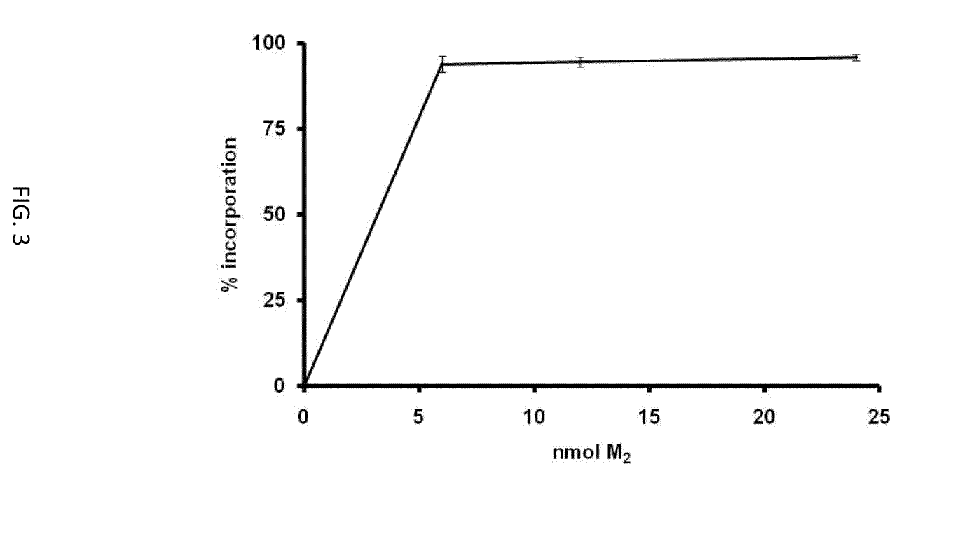

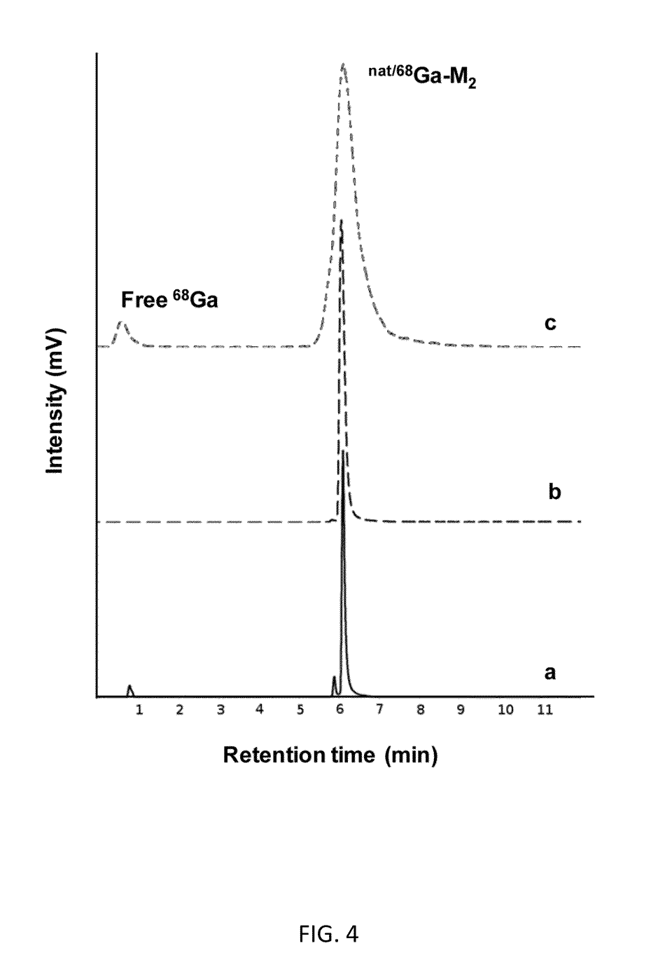

M₂ was radiolabeled with ⁶⁸Ga using the fractionation method and radiochemical purity (RCP) of >95% were achieved within 10 min, with 13 min selected as the optimal heating time. FIG. 3 shows that ⁶⁸Ga-M₂ is formed with high labeling efficiency over the range of peptide amounts investigated. Similar RCP were observed between the peptide amounts tested, therefore, 6 nmol (15 μg) was selected for radiolabeling experiments to achieve the highest specific activity and reaction conditions were optimized using 0.1 N NaOAc buffer. Co-injection of ^{nat}Ga-M₂ and ⁶⁸Ga-M₂ on HPLC showed excellent correlation between the UV, fluorescent, and radiometric peaks (FIG. 4). The log P value for ⁶⁸Ga-M₂ was calculated to be −2.09±0.02.

### Example 4

### Stability Studies

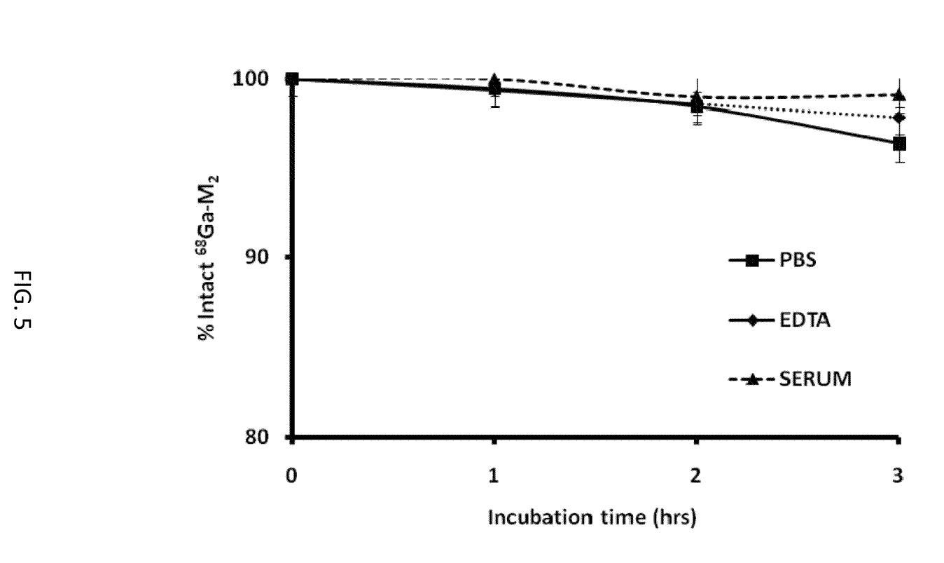

Peptide and optical stability were evaluated in water and PBS at 4° C. and room temperature. Over the 14 day incubation period, HPLC analysis revealed no significant degradation of peptide or fluorescent signal. Retention times were consistent and peak values did not significantly change based on quantification from the HPLC fluorescence detector. The effects of Ga-labeling conditions on the fluorescent properties of M₂ were evaluated using radioactive and ^{nat}Ga and showed no deterioration in fluorescence signal of the dual-labeled agent. ⁶⁸Ga-M₂ was stable in PBS, DTPA and mouse serum as indicated by >95% radiochemical purity at 3 h incubation (FIG. 5).

### Example 5

### Fluorescent Properties

Table 1 shows a summary of the spectral properties of IRDye 800CW, M₂ and ⁶⁸Ga-M₂. The optical spectrum of M₂ exhibited an absorbance maximum at 785 nm with an average extinction coefficient of 160,530±3802 M⁻¹ cm⁻¹ which is similar to that calculated for IRDye800. Similarly, ⁶⁸Ga-M₂ showed maximum absorbance at 785 nm but reported a lower extinction coefficient value of 108,069±7,918 M⁻¹ cm⁻¹. Using an excitation wavelength of 785 nm, M₂ and ⁶⁸Ga-M₂ demonstrated fluorescence quantum yields (0) of 0.034 and 0.031, respectively, relative to ICG. These values were in reasonable agreement with the calculated quantum yield of IRDye 800CW (Φ=0.034) and attests to the efficiency of these IRDye800-based peptide conjugates.

TABLE 1
Spectral properties of NIR and dual-labeled agents.

| | Excitation | Emission | Extinction | |
|---|---|---|---|---|
| | wavelength | maximum | coefficient | Quantum |
| Sample | (nm) | (nm) | (M⁻¹cm⁻¹) | yield |
| IRDye 800CW | 785 | 830 | 181,458 ± 2,189 | 0.034 |
| M₂ | 785 | 830 | 160,053 ± 3,802 | 0.034 |
| ⁶⁸Ga-M₂ | 785 | 830 | 108,069 ± 7,918 | 0.031 |

The fluorescence quantum yield (0) was measured using an aqueous solution of ICG (Φ=0.016). Data presented as mean±standard deviation (n=4).

### Example 6

### In Vivo Imaging

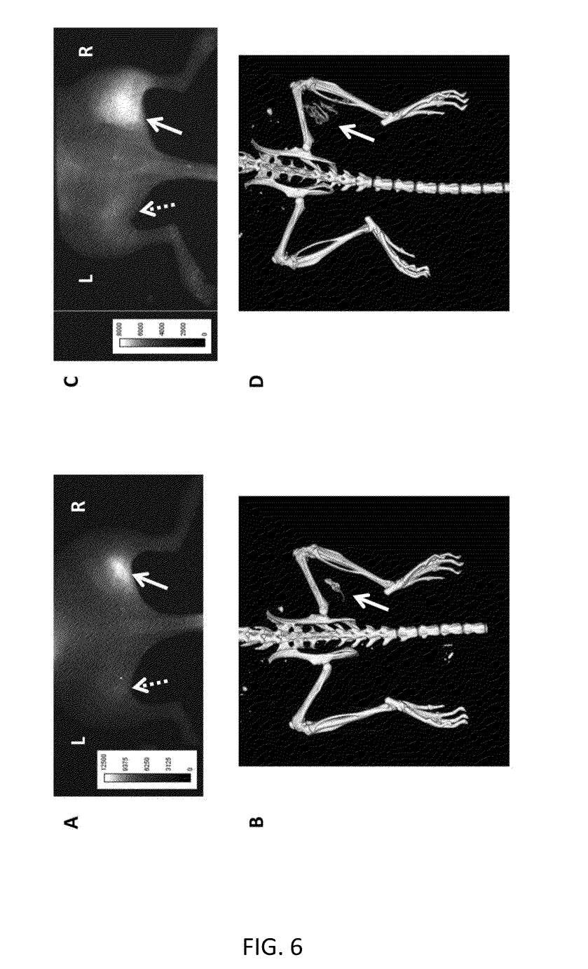

FIGS. 6A and 6C show typical NIR fluorescence images of the dorsal view of mice with right hindlimb injected with AdBMP2 cells (solid arrows) and the left hindlimb injected with Adempty cells (dashed arrows) taken at 4 days after implantation. Localization of ⁶⁸Ga-M₂ was observed in the tissue region with BMP-2 producing cells with minimal fluorescence detected in the contralateral region. FIGS. 6B and 6D show CTs acquired for each mouse on day 11 post-implantation and provide evidence of new bone formation at the site corresponding to agent uptake on the NIR images. The findings demonstrate the feasibility of NIR imaging following ⁶⁸Ga-labeling with consistent fluorescent signal obtained in all cases, and also indicate that the mechanism of tracer accumulation may be related to MMP-9 expression during HO.

In contrast with previous results using ⁶⁴Cu-M₂ (Rodenberg et al., 2010), PET imaging of ⁶⁸Ga-M₂ was confounded by the overwhelming signal from the nearby bladder and showed only negligible uptake at the target site. Nonetheless, mouse imaging shows that in vivo NIR signal and targeting of MMP-9 was not perturbed by ⁶⁸Ga labeling.

### Example 7

### Significance of Certain Embodiments of the Invention

The use of hybrid imaging has gained acceptance clinically led by the advent and utility of combining the functional imaging of nuclear imaging with anatomical correlation by CT, and more recently with the introduction of PET/MRI. In the case of cancer, multimodal imaging platforms have improved diagnosis through co-registration of images, providing physicians with methods to identify lesions with better certainty and accuracy and tailor treatment strategies (Poeppel et al., 2009; Cronin et al., 2010). As a result, tumors can be detected earlier and therapeutic intervention can be initiated prior to reaching advanced stages of the disease. An even further extension of multimodal imaging applications is intraoperative use for image-guided surgery by combining PET/NIR into a single agent. For example, an agent can be dual-labeled with a radionuclide and a NIR fluorophore and injected a day prior to surgery for PET/CT imaging for lesion detection and surgical planning. On the following day, the radioactivity will have decayed and the surgeon can remove the tumor using conventional methods, but could now incorporate intraoperative optical imaging to detect any existing NIR signal from residual tumor tissue. This directly permits visualization of any positive margins that still remain via molecular imaging and guides the surgeon on the potential need for further surgical intervention in real-time.

Because the sensitivities of CT and MRI are far lower than nuclear modalities, the design of agents bearing beacons for both nuclear and anatomical imaging is challenging. Conversely, optical imaging possesses similar sensitivity to nuclear imaging and the feasibility of a multimodality imaging approach with dual-labeled nuclear/NIR peptides has been described in a recent review (Kuil et al., 2010). Dual-labeling of antibodies with a radionuclide and a NIR fluorophore can be achieved using various combinations of IRDye 800CW and metal chelates with either ¹¹¹In or ⁶⁴Cu (Sampath et al., 2008; Sampath et al., 2007; Sampath et al., 2010). In the case of antibodies, it is known that extended plasma circulation times are critical for therapy as they reduce the need for frequent dosing. However, for imaging this creates high levels of radioactivity present in the blood and liver and mandates later imaging time points to achieve lower background levels and sufficient contrast. Longer-lived radionuclides such as ¹¹¹In and ⁶⁴Cu allow for delayed imaging and clearing of the agent from circulation. Thus, these radiometals are widely-used for antibody imaging and have been adopted in many dual-labeling strategies.

Peptides are of particular importance in molecular imaging due to favorable pharmacokinetic properties, specific interaction with cell surface receptors, preparation in high specific activities, and robust manufacturing schemes by solid phase synthesis. The ability of peptides to rapidly associate with receptors and clear from non-target sites permits imaging at early time points through the use of shorter-lived radionuclides such as ⁶⁸Ga. The use of ⁶⁸Ga-peptides has experienced tremendous growth over the past decade (Hofmann et al., 2001; Maecke et al., 2005; Virgolini et al., 2010). Since previous reports with dual-labeled nuclear/NIR agents predominantly used ⁶⁴Cu or ¹¹¹In, both of which have milder radiolabeling conditions (pH >5, heating temperatures RT-80° C.) than ⁶⁸Ga (pH 3.5-4, heating temperatures 80-98° C. for DOTA chelates), the feasibility of a dual-labeling strategy using ⁶⁸Ga and assessment of the effects of labeling conditions on the optical properties of IRDye 800CW was examined. Multiple ⁶⁸Ga labeling schemes have been reported that minimize the effect of ⁶⁸Ge breakthrough and concentrate the generator eluate through fractionation or ion exchange methods, but a common thread shared by most DOTA-based schemes is the need to label in acidic conditions with elevated heating (Velikyan et al., 2004; Breeman et al., 2005; Zhernosekov et al., 2007; Meyer et al,. 2004). Thus, these effects were explored on M₂ to evaluate chemical and optical stability in response to ⁶⁸Ga labeling using a modified version of the method described by Breeman et al (2005).

The two week stability study of M₂ revealed no degradation of peptide or fluorescent signal at 4° C. or room temperature. Initially, care was taken to shield samples from light, but the data showed no added benefit as samples exposed to ambient light showed identical fluorescent intensity by HPLC analysis. The stability of the dye was further confirmed when M₂ was labeled with ⁶⁸Gar/^{nat}Ga and showed no alteration in fluorescence profile. Optical properties were assessed for IRDye 800CW, M₂ and ⁶⁸Ga-M₂ and showed minor changes in extinction coefficient and quantum yield in response to conjugation and radiolabeling, indicating the ability of the dye to withstand synthesis conditions. A somewhat lower extinction coefficient was observed for ⁶⁸Ga-M₂ and may be attributable to the presence of acetate buffer in the reaction mixture post-radiolabeling, in contrast to the other samples that were analyzed in water. Stability studies looking at the radiochemical stability also served as opportunities to confirm fluorescence signal in response to the effects of ⁶⁸Ga labeling over extended periods of time.

The conserved fluorescent yield of the NIR fluorophore indicated no significant changes under any of the test conditions and allowed one to proceed to a model of HO where targeting specificity and fluorescent properties of ⁶⁸Ga-M₂ were assessed in vivo. Qualitatively, a high degree of similarity between pattern and location of new bone formation was observed on both NIR and CT images. Because of the inherent variability of the transduced cell implantations, various degrees of new bone formation were present, but in each case the anatomic rendering of new bone by CT corresponded to the location and relative shape of the functional image from NIR fluorescence. The mouse in FIGS. 6A,B had a smaller bone mass as shown by the day 11 CT, whereas the mouse in FIGS. 6C,D had much larger bone formation. Variation in size was evident as early as day 4 by NIRF imaging and preceded the anatomical appearance of bone by CT. PET/CT images showed poor accumulation of ⁶⁸Ga-M₂ at sites of HO, possibly due to sub-optimal pharmacokinetic properties. The tracer was rapidly cleared from circulation via the kidneys and had high bladder activity. No non-specific binding was observed. Conversely, earlier studies using ⁶⁴Cu-M₂ had completely different biodistribution as evidenced by much higher background levels and liver and gut uptake at early time points (<18 h), but could clearly delineate new bone formation by PET/CT (Rodenberg et al., 2010). The discrepancies between the PET/CT findings of both agents may also be attributed to changing of the radiometal from ⁶⁴Cu to ⁶⁸Ga. A study evaluating various somatostatin octapeptides radiolabeled with ⁶⁷¹⁶⁸Ga, ¹¹¹In, and Yttrium-90 (⁹⁰Y) found that changing the radiometal does indeed cause variation in receptor-binding affinities, cellular internalization rates, hepatic clearance, and in vivo pharmacology (Antunes et al., 2007). Further optimization of ⁶⁸Ga-M₂ is needed to obtain better target visualization by PET/CT, but the in vivo findings demonstrate the feasibility of generating and applying a dual-labeled probe with ⁶⁸Ga and NIRF for multimodality PET/NIRF imaging.

Thus, certain embodiments of the invention encompass a dual-labeled MMP-9 targeting peptide using ⁶⁸Ga and IRDye800. The addition of multiple reporters to a targeting agent has the ability to enhance each modality, but also requires additional testing and validation to ensure the agent is stable across biologic, chemical, radiochemical and optical criteria; such testing is routine, however. The exemplary dual conjugate in this study showed excellent stability and retention of optical properties. ⁶⁸Ga labeling methods were developed and optimized to yield a dual-labeled peptide for PET/NIRF imaging that had excellent radiochemical stability. This study showed that ⁶⁸Ga labeling conditions did not adversely affect the optical properties of ⁶⁸Ga-M₂ and that this strategy can be applied to other dual-labeled peptides and other NIR fluorophores. One can optimize the pharmacokinetic properties of ⁶⁸Ga-M₂ for PET/CT/NIRF imaging of HO and other disease models.

### Example 8

### MMP-9 as a Biomarker of Heterotopic Ossification

Heterotopic ossification (HO) is a serious disorder that occurs when there is aberrant bone morphogenic protein (BMP) signaling in soft tissues. Currently, there are no methods to detect HO before mineralization occurs. Yet once mineralization occurs, there are no effective treatments to reverse HO. Herein, we used confirmatory ex vivo tissue analyses and in vivo molecular imaging of an established murine animal model of BMP-induced HO to show that MMP-9 can be detected as an early-stage biomarker prior to mineralization. Ex vivo analyses show active MMP-9 protein is significantly elevated within tissues undergoing HO as early as 48 hours after BMP induction, with its expression co-localizing to nerves and vessels. In vivo molecular imaging with a dual-labeled near-infrared fluorescence and μPET agent specific to MMP-2/-9 expression paralleled the ex vivo observations and reflected the site of HO formation as detected from μCT seven days later. The results indicate that the MMP-9 is a biomarker of the early extracellular matrix (ECM) re-organization and could be used as an in vivo diagnostic for detecting HO or conversely for monitoring the success of tissue-engineered bone implants that employ ECM biology for engraftment.

### Example 9

### Specific Embodiments of the Invention

Heterotopic ossification (HO) is endochondral bone formation at non-skeletal sites that often results from inappropriate BMP signaling in soft tissues. The disease can be initiated by traumatic injury to the muscle and soft tissues (Clever et al., 2010), altered blood flow in vessels (Yao et al., 2007), and through genetic mutation in the BMP-type 1 receptor (for review see (Shore and Kaplan, 2010)). Although HO affects less than 10% of the general population, for those affected, it can have devastating outcomes (Shore and Kaplan, 2010). Recent statistics indicate that the incidence among the military population is significantly higher, with approximately 60% of all traumatic injuries resulting in substantial HO (Forsberg et al., 2009). It has been speculated that this disparity is due to the types of traumatic injuries suffered from exposure to improvised explosive devices (IEDs), which involve substantial change in both the nervous system and soft tissues as compared to crush injuries or amputations within the general population. To date, there are no effective inhibitors of the bone formation, presumably due to the difficulty in identifying the HO prior to the deposition of mineralized matrix. Clinical detection of HO is currently performed with computed tomography (CT) or bone scans with 99mTc-MDP.

These diagnostic imaging modalities rely on the presence of bone formation that occurs at a time when the HO process is in advanced stages and cannot benefit from therapeutic intervention. Surgical removal of mineralized tissues may have limited benefit, since HO regrowth is often more robust than its original onset. Consequentially, better diagnostics are essential for detecting and dissecting the biology associated with the tissue remodeling that occurs with HO. Better diagnostics could enable development of effective therapeutic strategies to halt HO progression. Conversely, the strategies to detect and inhibit HO may also provide insights to monitor and develop new tissue-engineering strategies for functional bone replacement.

There is a murine model of BMP-induced HO and early tissue remodeling stages involve regional stem-progenitor cell recruitment for chondro-osseous differentiation followed by new vessel formation and the rapid remodeling of the vasculature that occurs simultaneously with the generation of brown adipose (Olmsted-Davis et al,. 2007). These early stages are thought to prepare the microenvironment for progenitor recruitment for cartilage (Shafer et al., 2007) and bone as well as define the location and boundaries of the HO itself (Ortega et al., 2004; Koivunen et al., 1999; Kuhnast et al., 2004). Once cartilage matrix is produced, it is degraded by matrix metalloproteinase-9 (MMP-9) and MMP-13 (Ortega et al., 2004) for replacement with new osteoid. Thus, in certain embodiments of the invention the MMPs are a useful target for diagnostic molecular imaging to detect early HO disease on the basis of early tissue remodeling processes.

From screening of peptide libraries, Koivunen et al. identified a cyclic CTT peptide c(CTTHWGFTLC) with potent inhibitory activity against MMP-2/9 that arises from the HWGF peptide motif (Koivunen et al., 1999). However, the CTT peptide is highly susceptible to non-specific degradation thus limiting its potential as an in vivo imaging agent (Kuhnast et al., 2004; Sprague et al., 2006). Using structure-activity relationship to create an optimized HWGF peptide motif, Wang et al (2010) created the cyclic peptide c(KAHWGFTLD)NH2 to which the inventors added a lysine to the N-terminus for conjugation of an NIR fluorescent dye to detect early HO from both NIR and WET. NIR fluorescence provides a fast and simple non-radioactive means for imaging (for review see (Sevick-Muraca et al., 2008) but because it is not yet validated for quantitative imaging, the inventors dual labeled for μPET quantification.

Embodiments of the invention provide molecular imaging in a murine model to show increased active MMP-9 protein expression in vivo immediately after BMP-induced induction of HO, but before mineralization occurs. Complimentary ex vivo data that shows MMP-9 expression is associated with a number of tissue structures undergoing remodeling to support new bone formation. Micro-computed tomography (₁CT) was used to later visualize mineralization at the site of NIR and μPET detection of active MMP-9. The study was designed to determine whether MMP-9 could provide a tentative target for diagnosing ECM changes prior to mineralization and potentially enable the earliest intervention prior to bone matrix formation.

### Example 10

### Exemplary Materials and Methods

Animal Procedures

Human fibroblast (MRCS) cells (American Tissue Type Culture Collection, Manassas Va.) transduced with either Adempty or AdBMP2 (for details see Gugala et al,. 2003 or Fujimoto et al., 2008) were washed with PBS, removed with trypsin and resuspended at a concentration of 5×10⁶ cells per 100 μL of PBS. An intramuscular injection of 50 μL into each hind limb quadriceps muscle of nonobese diabetic/severely compromised immunodeficient (NOD/SCID) mice was performed. Control transduced cells were injected into the left limb and BMP2 transduced cells were delivered into the right limb of each animal. For ex vivo evaluation of RNA protein expression and active gelatinase protein content, animals were euthanized at time points of 1, 2, 3, 4, 5, and 6 days post injection. Hind limbs were harvested and tissues were placed in formalin, or frozen for subsequent analyses (as described below). For in vivo evaluation of mice were imaged day 2, 4, or 6 post-implantation with μPET, μCT and NIR and seven days later, μCT imaging was conducted to assess mineralization (as described below) before the animals were euthanized. For all imaging procedures, mice were anesthetized with 1% isoflurane. All animal studies were performed in accordance with the standards of Baylor College of Medicine (Houston, Tex.), Department of Comparative Medicine and The University of Texas Health Science Center (Houston Tex.), Center for Molecular Imaging after review and approval of the protocol by their respective Institutional Animal Care and Use Committee (IACUC) or Animal Welfare Committee (AWC).

Histology

Mouse hind limbs were formalin fixed, decalcified, divided in half longitudinally to expose the internal tissues, then both halves of the tissue embedded into a single paraffin block or alternatively snap frozen for sectioning. The tissues were oriented so that the internal areas were exposed to the outside of the paraffin block, allowing for the tissue to be sectioned from the inside out. Serial sections (5 μm) were prepared that encompassed the whole hind limb reactive site (approximately 10-15 sections per tissue specimen depending on the type of transduced cells the tissue received). Hematoxylin and Eosin staining was then performed on every 5th slide to locate the center region containing either the delivery cells or the newly forming endochondral bone.

Serial unstained slides were used for immunohistochemical staining (either single or double-antibody labeling). For double antibody labeling, samples were treated with both primary antibodies simultaneously followed by washing and incubation with respective secondary antibodies, used at 1:500 dilution to which Alexa Fluor 488, 594, or 647 (Invitrogen by Life Technologies, Carlsbad, Calif.) were conjugated. Briefly, sections were fixed with 4% paraformaldehyde, PBS washed and treated with 0.25% Triton X-100 in Tris-buffered saline (19.98 mM/L Tris, 136 mM/L NaCl, pH 7.4). The Mouse on Mouse (M.O.M.) kit for detecting mouse primary antibodies on mouse tissue (Vector Laboratories, Burlingame, Calif.) was applied to the sections according to manufacturer's protocol. A goat anti-mouse MMP-9 antibody (R&D Systems, Minneapolis, Minn.) was used at a 1:150 dilution, anti-neurofilament mouse monoclonal antibody used at 1:150 dilution (Sigma Chem Co, St. Louis, Mo.), and anti-von Willibrand Factor (VWF), rabbit polyclonal antibody was used at 1:300 dilution (Chemicon-Millipore, Billerica, Mass.). Slides were then covered with mounting medium containing the nuclear stain DAPI (Vector Labs). Stained tissue sections were examined by confocal microscopy (Zeiss Inc, Thornwood, N.Y., LSM 510 META) using a 20×/0.75 NA objective lens.

Ex Vivo Analysis of Protein Content

Q-RT-PCR (Real Time PCR)

From the harvested muscle tissue surrounding the injection site of either control or BMP2 transduced cells, total RNA was collected using a Trizol reagent (Life Technologies, Carlsbad, Calif.). RNA integrity was confirmed by agarose gel electrophoresis. cDNA was synthesized from RNA using the RT2 first strand kit (SA Biosciences Inc, Frederick, Md.). The cDNA from each sample was analyzed separately, the results were averaged and standard error of the mean calculated. The cDNA from muscles with control or BMP2 transduced cells were subjected to qRT-PCR analysis in parallel using a 7900HT PRISM Real-Time PCR machine (Applied Biosystems, Carlsbad, Calif.). The Ct values were normalized to both internal 18S ribosomal RNA used in multiplexing and to each other to remove changes in gene expression common to both the control and BMP-2 tissues by using the method of ΔΔ Ct along with SYBR Green probes and qPCR primers (SABiosciences, Frederick, Md.). The analyses were conducted in triplicate for 8 biological samples at each time point and were reported as the average and standard deviation of the fraction of protein RNA that was attributed to MMP-9 RNA. Significance was determined by standard T-test.

Quantification of Active MMP 2 and 9 Protein

Protein extracts were prepared from the muscle surrounding the site of injection of either BMP2-producing or control cells the Total Protein Extraction Kit (Millipore, Billerica, Mass.). Briefly, tissues (n=8 animals) were homogenized separately and protein extracts centrifuged according to kit instructions. The resultant protein concentrations were determined using a Bio-Rad Protein Assay Kit® (Bio-Rad Corp, Hercules, Calif.) and samples were then analyzed for both active protein using MMP-2 and MMP-9 Biotrak Activity Assay System (GE Healthcare, Piscataway, N.J.) and total protein using the MMP9 protein standard provided by the manufacturer (R&D Systems) according to manufacturer's protocol. Sample analysis was done in duplicate, and the final values were calculated as the fraction of total active protein within the tissue associated with MMP-9, as the average and standard deviation. Significance was determined by standard T-test.

Synthesis and Validation of Dual Labeled In Vivo Imaging Agent Against MMP-9

The present invention concerns specific imaging of gelatinases separately using pPET and NIR technologies in order to follow the in vivo changes in active MMP-9 in tissues in the early stages of HO. Therefore, the inventors specifically developed and validated a molecular imaging agent specifically for the study of HO. The following describes the synthesis and validation process for utilizing the imaging agent in trace dosages for early detection of HO on the basis of MMP-9 expression.

Reagents

All reagents were purchased from commercial sources and used without further purification. Chelex-100 resin was purchased from Bio-Rad Laboratories (Richmond, Calif.) and used with all aqueous buffers to ensure metal-free conditions. 64Cu was obtained from Washington University (St. Louis, Mo.) in the form of high-specific-activity 64CuCl2 in 0.05 M HCl. The MMP-targeting peptide M¹ [Lac(2,10)]DOTA-KKAHWGFTLD was synthesized by New England Peptide (Gardner, Mass.) according to standard Fmoc-protocols. Analytical high-performance liquid chromatography was performed on a Hitachi LaChrom system equipped with a 2.6 μm Kinetex C-18 column (Phenomenex, Torrance, Calif.) with a mobile phase of A=0.1% TFA in H₂O, B=0.1% TFA in CH3CN; gradient, 0 min=5% B, 45 min=100% B; flow rate, 1 mL/min. Radio-TLC was carried out on a AR-2000 scanner (Bioscan, Washington, D.C.) using instant thin-layer chromatography (ITLC) strips and 1:1 methanol/0.1 M ammonium acetate. Molecular weight measurement was carried out ESI on a Waters UPLC system equipped with a Waters PDA detector and a Waters TQD mass spectrometer.

Preparation of Imaging Agent

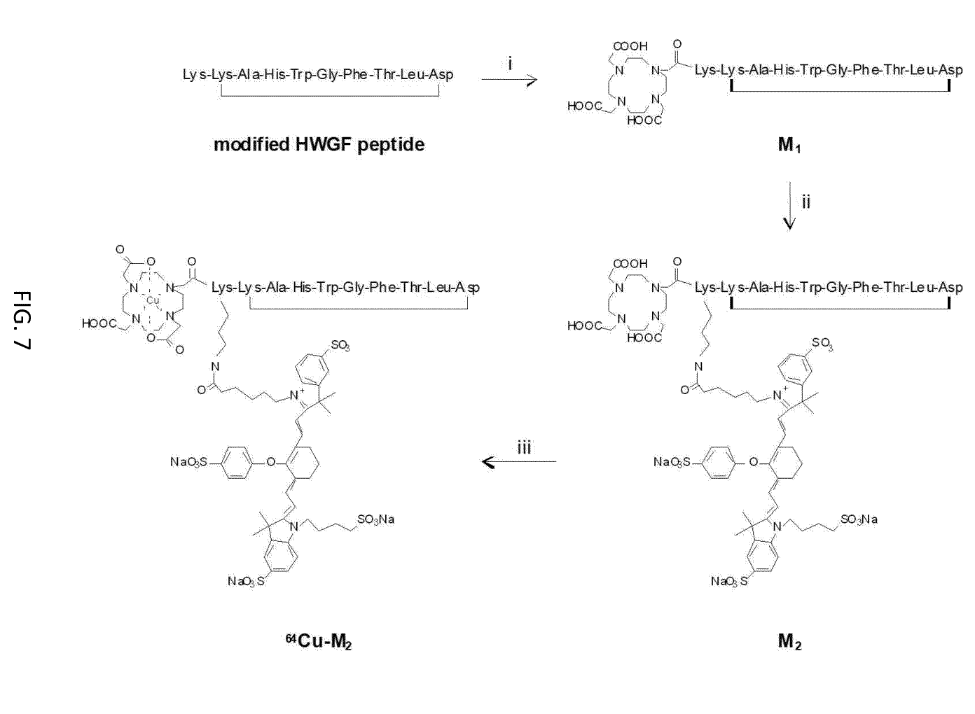

FIG. 7 shows the synthesis scheme for preparing the dualconjugated peptide. DOTA was coupled to [Lac (2,10)]KKAHWGFTLD (SEQ ID NO:1) on solid phase peptide synthesis to yield the conjugate M₁. M₁ (1 mg, 637 nmol) was dissolved in 500 μL 0.1 M sodium phosphate buffer, pH 8.33. IRDye800CW-NHS was added to the peptide conjugate at a 1:1 molar ratio and placed on a rotating mixer at 4° C. overnight. The sample was protected from light. The reaction mixture was loaded onto a 2000 MWCO spin column, centrifuged for 45 minutes at 3000 g and washed 3× with 500 μL of MilliQ water. The flow through was discarded. The column was then inverted and centrifuged at 3000 g for 5 mins, and the purified product (M₂) was collected, dried and weighed to determine yield. Samples were protected from light and stored at −20° C. for further use.

Radiochemistry

⁶⁴CuCl₂ was received in a small volume of 0.5 M HCl and diluted in 100 μl of 0.1 M sodium acetate to pH 6. For radiolabeling, 1-2 mCi of ⁶⁴CuCl₂ was added to 6-35 nmol of M₂ and the samples were incubated at 50° C. for 1 hr. Radiochemical purity was assessed by radio-TLC (Rf free Cu=0; Rf ⁶⁴Cu-M₂=0.9) and confirmed by radio-HPLC. 64Cu-M₂ was diluted in PBS and passed through a 0.22 μm syringe filter for in vivo studies.

Stability Studies

Since the CTT peptide is highly susceptible to non-specific degradation in vivo thus limiting its potential as an imaging agent (Kuhnast et al. 2004; Sprague et al,. 2006), we sought to assess stability of ⁶⁴Cu-M₂ in PBS, with a 500-fold excess DTPA solution, and in mouse serum. After radiolabeling, 150 μl of ⁶⁴Cu-M₂ was diluted in equal volumes of PBS or DTPA solution and kept at room temperature. Aliquots were taken at 0, 2, 6 and 24 hrs post-incubation and analyzed by radio-HPLC. To evaluate serum stability, 150 μl of ⁶⁴Cu-M₂ was added to 150 μl of 50% mouse serum and incubated at 37° C. An 80 μl aliquot was taken at each of the above-mentioned time points and added to 160 μl ice-cold acetonitrile. The samples were centrifuged at 14,000 g for 5 min and the supernatant was collected and analyzed by radio-HPLC.

Gelatin Zymography

Biological activity of conjugates or inhibition of MMP-9 by 1, M2 was examined by zymography against inhibitory control peptide CTT. 10 μg of CTT, M1, M2 were incubated with MMP-9 (AnaSpec, Fremont, Calif.) at room temperature for 2 hours and then electrophoresed in 5.0% SDS-PAGE containing 0.01% gelatin (a 5.0% SDS-PAGE without gelatin is used as a stacking gel). The sample was then re-natured in 2.5% Triton X-100 for 2 hours at room temperature then incubated at 37° C. for 2 hours in buffer containing 50 mM Tris (pH 7.4), 150 mM NaCl and 10 mM CaCl₂. The gel was stained using 0.25% Coomassie blue; destaining was performed in a methanol:water:glacial acetic acid (45:45:10) mixture for 20-60 minutes. Clear bands indicated enzymatic activity and the percentage of inhibition of M₁ and M₂ relative to the CTT control peptide was reported as the fraction of Coomassie staining intensity relative to the CTT control peptide.

In Vivo Molecular Imaging

μPET/CT Imaging

To visualize the in vivo distribution of the radioisotope on the dual-labeled peptide, μPET/CT imaging was performed using a Siemens Inveon μPET/CT scanner (Siemens Medical, Knoxville, Tenn.). The CT imaging parameters were an x-ray voltage of 80 kV with an anode current of 500 μA and an exposure time of 260 milliseconds of each of the 120 rotation steps over the total rotation of 220° at low system magnification. After μCT imaging, μPET emission scans were performed with 5 min acquisition times. μPET and μCT images were reconstructed using two-dimensional filtered back-projection and a Feldkamp cone-beam algorithm with a ramp filter cutoff at the Nyquist frequency, respectively. μPET and μCT image fusion and image analysis were performed using ASIPro and Inveon Research Workplace (Siemens Preclinical Solutions).

The anesthetized mice were injected intravenously with ⁶⁴Cu-M₂ (200 μCi, 6 nmol). μPET/CT images were acquired in the prone position at 6 and 18 hrs post-injection of ⁶⁴Cu-M₂. To confirm molecular specificity of the agent, blocking studies were performed in which 3 additional animals from the day 4 Adempty/AdBMP2 post-implantation group were injected with 200-fold excess of M₁ 24 hours prior to injection of ⁶⁴Cu-M₂ and 7 days later, μCT was performed. In all cases, images were acquired at 6 and 18 hrs post-injection of ⁶⁴Cu-M₂.

In Vivo Fluorescence Imaging

NIR fluorescence images were acquired using custom-built fluorescence imaging systems (Houston et al., 2005) 18 hours after intravenous administration of ⁶⁴Cu-M₂. Briefly, a field of view was illuminated with 785 nm of light from a laser diode, outfitted with a convex lens and diffuser to create a uniform excitation field. The fluorescence was collected through holographic and interference filters placed before a Nikon camera lens. The images were finally captured by an electron-multiplying charge-coupled device camera (PhotonMax 512; Princeton Instruments, Princeton, N.J.) with 200 to 400 milliseconds of integration time. For acquisition of white-light images, the optical filters were removed, and a low-power lamp illuminated the subject. Image acquisition was accomplished by V++ software (Aukland, New Zealand).

Data Analysis

To obtain the % injected dose per gram (% ID/g) of ⁶⁴Cu-M₂, ROIs were applied to coronal μPET images to determine local tracer concentration and were normalized by body mass (g) and total injected dose. Target-to-background ratios (T/Bs) from the μPET coronal projections were computed using the same numerical area on the contralateral limb to represent the background region. Target-to-background ratios (T/Bs) were similarly computed from the ventral NIR views.

### Example 11

### Histology and Immunohistochemical Staining

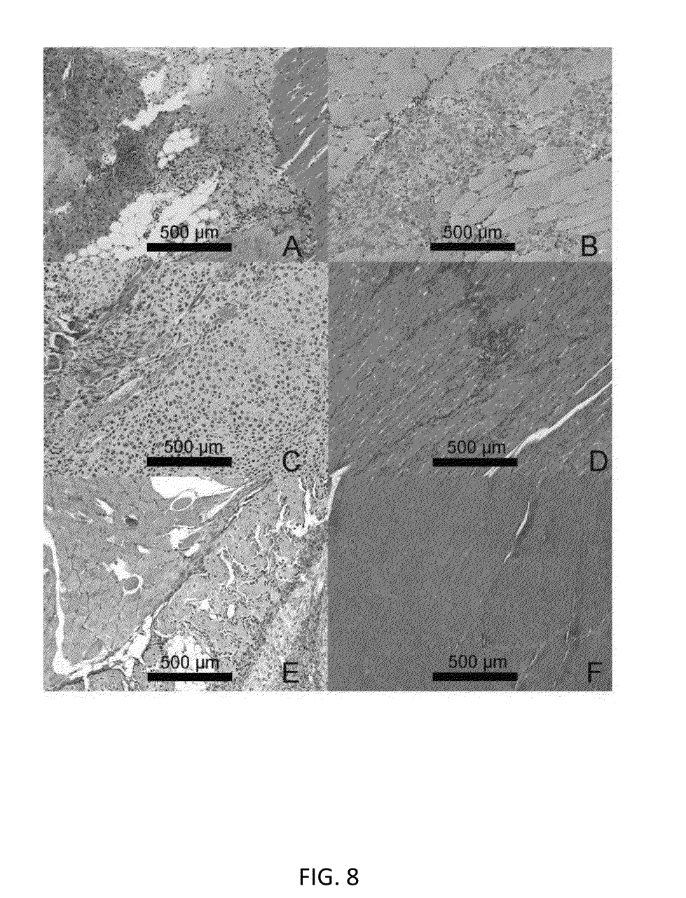

FIGS. 8 A, C, and E show H&E images of paraffin embedded sections of regional tissues 2, 6, and 10 days following AdBMP2 cells while FIGS. 8 B, D, and E show the corresponding images for Adempty control cells. In agreement with our prior studies (Olmsted-Davis et al., 2002; Gugala et al,. 2003). immediately following delivery of the transduced cells we observe a substantial cellular infiltration in response to the transduced cells regardless of the BMP2 expression (FIGS. 8A and B). The cellular response appears to wane in the control tissue region whereas the tissues receiving AdBMP2 transduced cells continue to have a large number of replicating cells. By day 6, cartilage appears within the tissues (FIG. 8C) while in controls it appears that the cellular reaction is almost completely gone (FIG. 8D). By day 10 there is substantial bone within the tissues receiving the AdBMP2 cells, whereas the tissue of animals receiving the Adempty transduced (control) cells appear similar to normal muscle (FIGS. 8E and F, respectively).

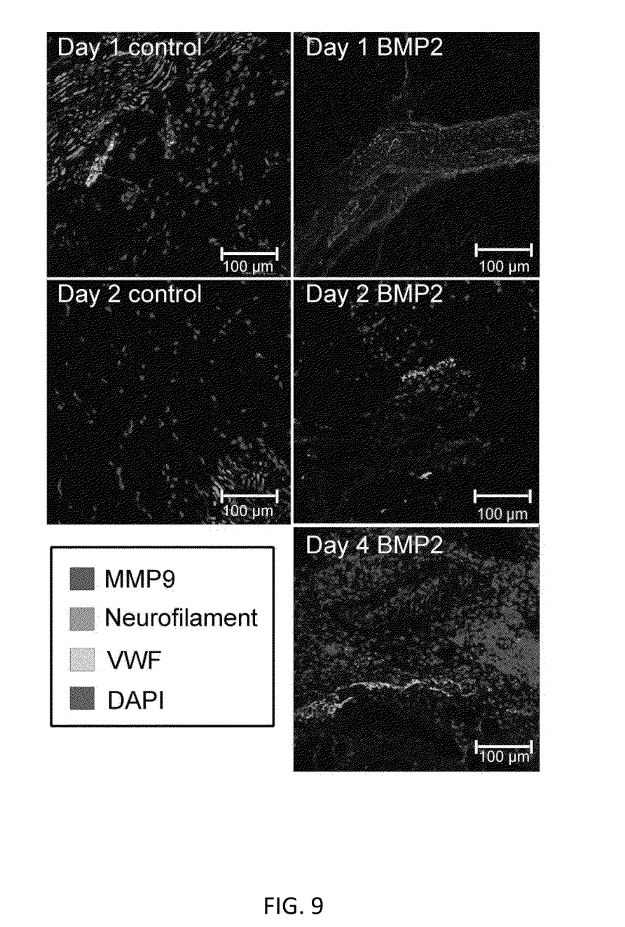

FIG. 9 shows that MMP-9 (red staining) was observed in tissues isolated 24 and 48 hours after delivery of AdBMP2 and Adempty transduced cells. However, no MMP-9 positive cell staining was observed within the tissues 72 hours after receiving Adempty (data not shown). MMP-9 expression (red) appeared to be associated with the nerve tissues (green staining) in the sample one day after receiving AdBMP2 transduced cells, whereas MMP-9 cell staining appeared to be uniformly dispersed within the control tissues. Examination of limbs injected with BMP2 on day 2 revealed nerve-associated expression similar to day 1, but also localization near von Willibrand factor (VWF) positive vasculature (yellow staining). This pattern was observed through day 4 and prior to the appearance of cartilage. Presumably, MMP-9 expression is associated with remodeling of the tissues at a point when progenitors are assembling to form the initial cartilage condensation. The timing of MMP-9 expression within the tissue appears to match those predicted by the RNA and protein analysis (as described below).

MMP-9 RNA and Protein Expression

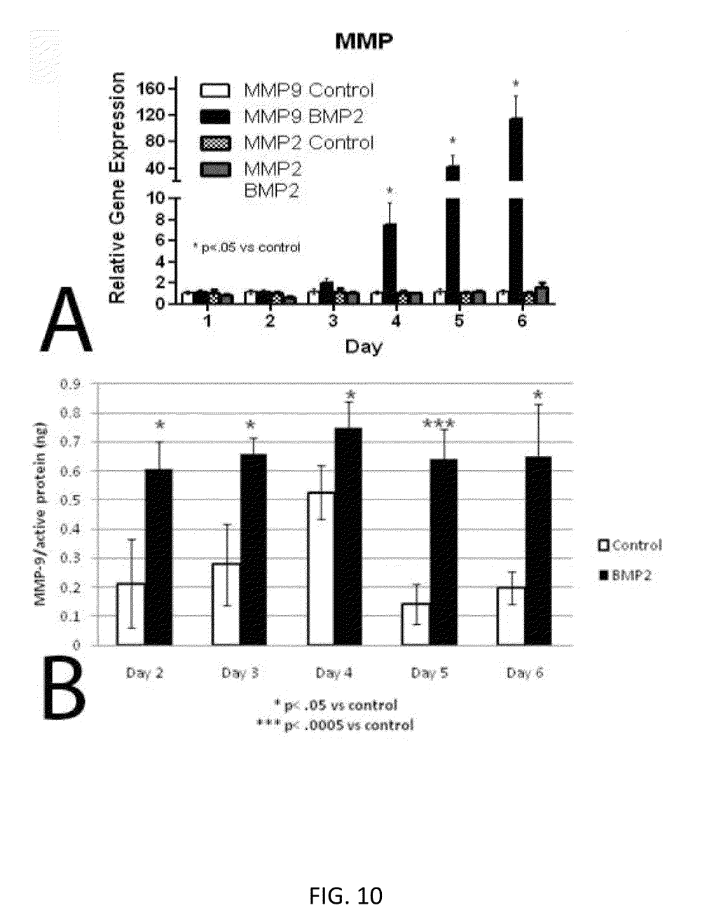

As illustrated in FIG. 10A, MMP-9 RNA in tissues receiving the AdBMP2 cells was significantly elevated starting 4 days after induction and continued to be significantly elevated (p<0.01) through the first appearance of heterotopic bone. In contrast MMP-2 RNA was not significantly elevated. Additionally, the expression of MMP-2 and -9 RNA within the control tissues suggested that there was low to undetectable levels of RNA in these samples.

Because the molecular imaging reagent is based upon an inhibitor of active MMP-9 (see below), the inventors measured amounts of MMP-9 using an ELISA-based system that detects only active MMP-9. The results shown in FIG. 10B indicates that protein extracts isolated from tissues receiving the BMP2 transduced cells have significantly more active MMP-9 protein, than those receiving control cells and that these elevated levels remained unchanged across the course of HO. The results collectively indicate that MMP-9 is activated by delivery of the BMP2-producing cells during all stages of endochondral bone formation. Further, this activation may be due to cleavage and utilization of stored MMP-9 protein, immediately following induction of HO, but is then rapidly replaced by newly synthesized MMP-9. Activated MMP-2 remained below the level of detection.

### Example 12

### Molecular Imaging Agent for MMP-9

Synthesis and radiolabeling of ⁶⁴Cu-M₂ Conjugation of IRDye800CW to M₁ was performed to yield the dual-conjugate M₂. Reaction yields were 35-40% and sample purity was >90% as confirmed by analytical HPLC showing a retention time of 6.1 min for M₂ compared to 5.9 min for M₁ from the 280 nm channel and a single retention peak at 6.1 min for M₂ from the 780 nm channel. There was no free dye in the sample as indicated by the lack of a peak at 5.4 min which corresponds to the retention time of IRDye800CW. Mass spectrometry showed 1278.93 [M+H]2⁺ and 2555.9 [M]⁺ and was in excellent agreement with calculated values. Radiolabeling with ⁶⁴Cu was achieved with high yield and purity as determined by radio-TLC and radio-HPLC. The presence of free copper by ITLC (R_{f}=0.9) was minor and confirmation by radio-HPLC routinely showed high sample purity (96.5±1.9%), therefore, the resulting radiotracer was used without further purification for in vivo studies.

Stability Studies

The in vitro stability of ⁶⁴Cu-M₂ was evaluated and is summarized in Table 2. Radio-HPLC analysis showed no peptide degradation at early (2 and 6 hrs) or delayed time points following incubation in PBS, with >98% of the sample still intact at 24 hrs post-mixing. Similarly, the DTPA challenge study did not result in a significant dissociation of copper from the radiolabeled complex as shown by the high radiochemical purity (RCP) of ⁶⁴Cu-M₂ (96.4±1.1%) at 24 hrs following incubation. Serum stability studies were performed in mouse serum and the sample showed excellent stability at 2 and 6 hours following incubation. However, a significant decrease in the RCP of ⁶⁴Cu-M₂ was observed at 24 hrs (53.7±3.7%). No loss or breakdown of the fluorescent peak associated with the peptide was noted in any of the experiments.

TABLE 2
In vitro stability of ⁶⁴Cu-M₂ with percentage of intact component
as a function of incubation media and time

| Incubation time (hrs) | PBS | DTPA | Serum |
|---|---|---|---|
| 2 | 99.7 ± 0.8 | 97.8 ± 1.3 | 99.6 ± 0.6 |
| 6 | 99.4 ± 0.7 | 97.7 ± 0.6 | 94.8 ± 0.6 |
| 24 | 98.9 ± 1.5 | 96.4 ± 1.1 | 53.7 ± 3.7 |

Data normalized to 100% at t=0 and presented as mean±standard deviation (n=3).

Zymography

Inhibition of MMP-9 by M₁ and M₂ was examined by zymography to determine the effect of DOTA and IRDye800CW conjugation. M₁ exhibited similar inhibition compared to the previously described CTT inhibitor. The presence of the IRDye moiety on M₂ resulted in a 10 fold decrease in inhibitory effect compared to M₁.

### Example 13

### In Vivo Imaging

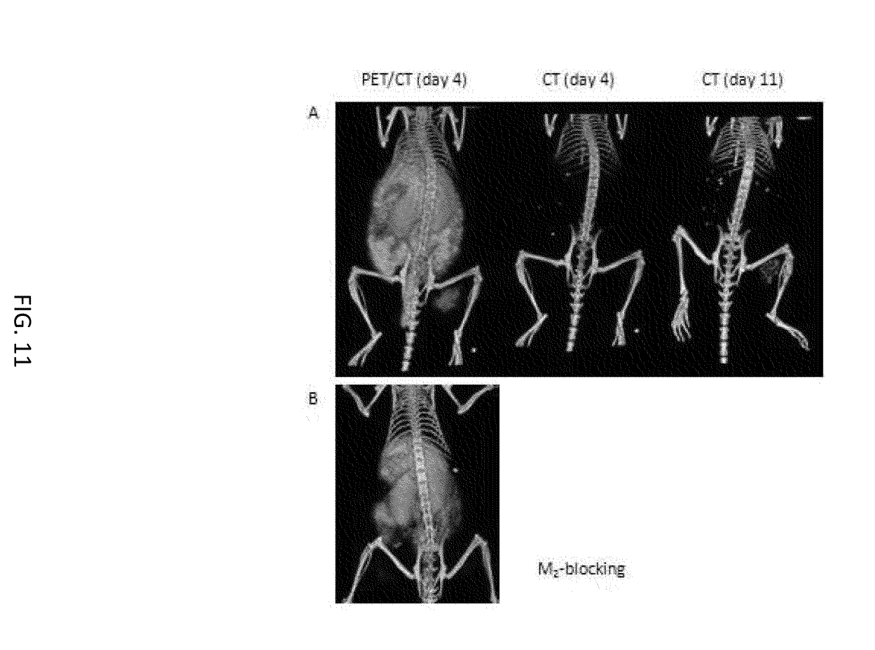

FIG. 11 shows coronal slices from the μPET/CT images taken at day 4 postimplantation of the bone putty in comparison with μCT taken at day 11 post-implantation. At day 4, localization of 64Cu-M2 at the sight of AdBMP2 transduced cell implantation is evident by μPET whereas the corresponding μCT scan does not show any indication of new bone formation. The follow-up μCT taken seven days later shows the ectopic bone and corresponds to the same region of μPET signal. Table 3 shows that T/B ratio computed from μPET and NIR as well as the % injected dose/gm in the region inoculated with AdBMP2 transduced cells were maximum at day 4 after AdBMP2 cell implantation, although the results were not statistically significant.

TABLE 3
Quantification of μPET/CT and NIR imaging results for mice
injected with ⁶⁴Cu-M₂.

| | Target/ | | |
|---|---|---|---|
| Days post- | background | % injected dose/g | Target/background |
| implantation | ratio (PET/CT) | (PET/CT) | ratio (NIR) |
| 2 | 3.29 ± 0.9 | 0.59 ± 0.1 | 1.13 ± 0.03 |
| 4 | 4.02 ± 1.39 | 0.61 ± 0.04* | 1.24 ± 0.05 |
| 4 with 200x M₁ | 1.44 ± 0.66 | 0.38 ± 0.04* | 1.13 ± 0.18 |
| as blocking | | | |
| 6 | 3.41 ± 0.51 | 0.59 ± 0.04 | 1.10 ± 0.03 |

*Indicates statistical significance from 4 hr blocking study (p < 0.05).

To confirm the specificity of tracer uptake for MMP-9, a blocking dose of M₁ was administered prior to imaging that successfully inhibited uptake of ⁶⁴Cu-M₂ at the site of tissue remodeling associated with AdBMP2 cell injection. As shown in Table 3, the T/B ratio computed from μPET was reduced from 4.02±1.39 to 1.44±0.66 following the blocking dose, while a lesser reduction was noted in NIR fluorescence, due presumably to changing tissue optical properties of the unvalidated approach. The quantification of % injected dose/gm showed statistically significant reduction in ⁶⁴Cu-M₂ uptake following 200× excess M₁. Bone formation was detected in these animals by μCT after another seven days, confirming that the trace dosage of ⁶⁴Cu-M₂ (and perhaps 200 fold greater dose of M₁) would not impact HO formation if MMP-9 activity were required for HO. The whole-body distribution of ⁶⁴Cu-M₂ showed slow clearance from circulation along with uptake in the liver and kidneys.

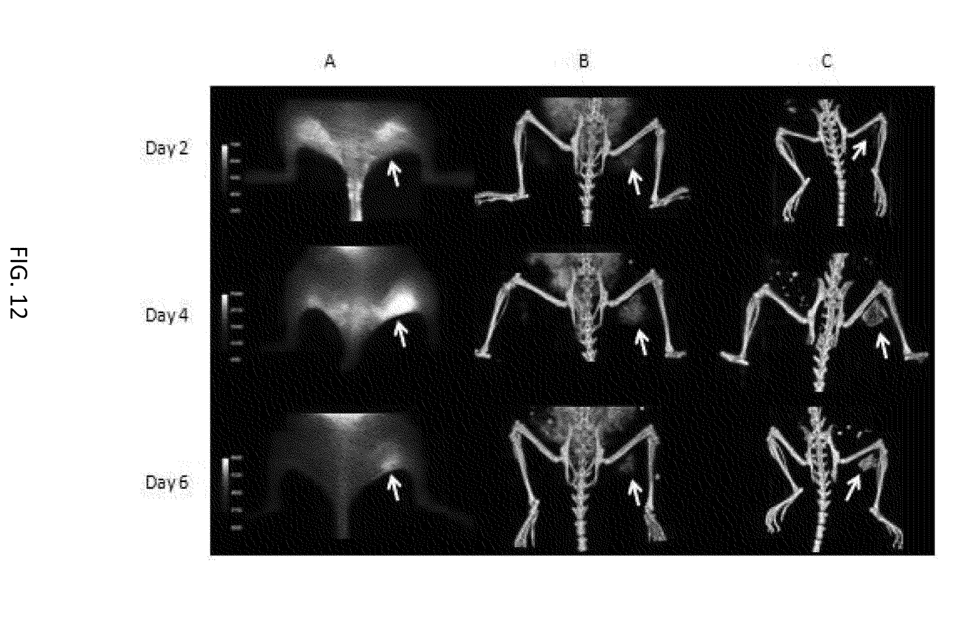

FIG. 12 shows typical NIR fluorescence images of the dorsal view mice with right hindlimb injected with AdBMP2 cells indicated by arrows and the left hindlimb injected with Adempty cells. At 2 days after injection, there is minimal expression of MMP-9 in both control and BMP2 sides; however maximal expression was observed in the tissue region with BMP-2 producing cells after 4 days. Expression diminishes at 6 days, indicating the transient expression of activated MMP-9. Also, NIR fluorescence could be detected in the contralateral region where a minor inflammatory response to the implantation was present at days 2 and 4.

Two days after injection of cells, the PET/CT scans (FIG. 12B) show moderate tracer accumulation with a T/B ratio of 3.29±0.9, reflective of uptake in the region of the control cell implantation and presumably due to inflammation. The T/B ratios at days 4 and 6 after inoculation of cells were 4.02±1.39 and 3.41±0.51, respectively and were not significantly different. ⁶⁴Cu-M₂ uptake observed by PET was confirmed by seven day follow-up CT images acquired on day 9, 11 and 13 post-implantation, respectively (FIG. 12C). The bone mass observed on the CT shows excellent correlation with the corresponding μPET/CT scan in terms of anatomical position, shape, and size. The data collectively indicates that detection of MMP9 can identify HO formation prior to radiological detection.

### Example 14

### Significance of Certain Embodiments of the Invention

This is the first demonstration of using the combination of in vitro and in vivo assays to find a diagnostic and potential therapeutic target of early HO prior to the appearance of cartilage and/or osteoid matrix. This technology is based on non-invasive μPET and NIR fluorescence imaging of the soft tissues to detect expression of MMP-9. Other imaging studies of tissue regeneration have used different labeled gelatinase inhibitors and activatable fluorescent agents to evaluate (i) the macrophage involvement in atherosclerotic plaques, (Fujimoto et al., 2008; Suzuki et al., 2008; Deguchi et al., 2006) and their response to experimental therapeutics, (Ohshima et al., 2010; Chang et al., 2010) (ii) the presence and expansion of abdominal (Razavian et al,. 2010) and intracranial aneurysms, (Kaijzel et al., 2010) (iii) the remodeling of cardiac tissues following myocardial infarct, (Chen et al., 2005) and (iv) cancer detection (Sprague et al., 2006). Indeed, MMPs contribute to all types of tissue remodeling, including the vasculature, and are often found associated with angiogenesis including that found during tumor formation.

MMPs are also involved in regulating the inflammatory response, through regulation of TGFβ (Sternlicht and Werb, 2001). MMPs are a family of proteases, which are formed as proproteins, and can be stored in an inactive form until further processed upon activation. This activation requires additional proteases, and often leads to additional regulation of their function. Inflammatory neutrophils (Ardi et al., 2007) have been shown to produce pro MMP-9, which is then activated by chymase from mast cells (Fang et al., 1997; Tchougounova et al., 2005).

The tissues staining positive for MMP-9 were taken immediately after induction (24 hrs) and appeared to be associated with the nerve. MMP-9 expression may be reflective of the nerve remodeling while control tissues were not nerve-associated but had a more generalized localization. One may speculate that the control tissues lacking BMP2 were not capable of undergoing the neuro-inflammatory response known to be induced by BMP2 (Salisbury et al, unpublished) and alternatively launched a different inflammatory response, which would not lead to bone and cartilage formation. An example of neuro-inflammation is the dysregulation of the neural stem cell pathways by neurologic inflammation in autoimmune encephalomyelitis and in multiple sclerosis (Wang et al., 2008). The immunohistochemical staining suggests neuro-inflammation as part of the early HO process. For example, the level of MMP-9 expression greatly dropped in the control tissues, with only a small nidus of MMP-9 positive cells clustered into a specific structure that were possibly involved in the removal of the Adempty transduced cells. However in the tissues receiving AdBMP2 cells, the MMP-9 positive cell staining appeared first at the nerve and subsequently throughout the region of new bone and cartilage. MMP-9 appears to be expressed by cells throughout the tissues by days 5-7 after induction, which is the time when cartilage matrix is present within the tissues. These results are consistent with the known role of MMP-9 and MMP-13 in endochondral bone formation through the degradation of cartilage matrix, for replacement with osteoid (reviewed in (Ortega et al., 2004)).

In the studies provided herein, the molecular imaging agent detects the activated form of both MMP-9 and MMP-2 as demonstrated by Koivunen, et al. (1999), Sprague, et al. (2006), and Wang, et al (2009) (as well as confirmed from gelatin zymography) and does not employ a cleavable peptide sequence for reporting (Chen et al., 2005). However, analysis of the tissues for both MMP-2 and MMP-9 activity suggested that the agent was probably detecting only MMP-9 since MMP-2 RNA and protein were undetectable. The fact that new bone formation was observed in all animals following AdBMP2 cell inoculation with corresponding ⁶⁴Cu-M₂ uptake suggests that at the trace doses administered, the inhibition of MMP-9 by the peptide was not sufficient to disrupt the process of HO. Indeed, animals receiving a 200-fold excess of M₁ in the blocking studies showed an expected decrease in ⁶⁴Cu-M₂ uptake, but still exhibited bone formation after 7 days as determined from μCT. While we did not assess the volume of bone formed to evaluate whether a reduction in HO occurred with dose of excess peptide inhibitor, a dose escalation study would be needed to assess the dose for therapeutic inhibition.

The positive μPET and NIR imaging signal within the tissues was detected as early as 2 days after induction and continued throughout the entire process of endochondral bone formation. These findings correlate with the ex vivo data showing active MMP-9 within the tissues, which was also found to be significantly elevated on all days. Interestingly, multiple animals from the day 2 group had notable tracer accumulation in the control region which then disappeared by day 4. The positive expression was not supported by our ex vivo quantification; however, we did observe elevated levels of MMP-9 protein within control tissues on day 1 which subsequently dropped. The result suggests that perhaps an initial, generalized inflammatory response was caused by the delivery of the adenovirus transduced cells, inducing transient MMP-9 expression within the control that could not be sustained in the absence of the BMP2 stimulus. The data suggests that the μPET analysis was even more sensitive at detecting MMP-9 expression within the tissues than either the ELISA or activity assays that were used for quantification. Since protein extracts were isolated from the entire tissue, considerable dilution may occur from inclusion of regions of the muscle not involved with the HO. Analysis of the tissues histologically shows a significant cellular response to the foreign cells, further supporting the imaging findings.

Although MMP-9 RNA expression is significantly elevated at the time of cartilage remodeling, just prior to bone matrix deposition, it is interesting that the μPET and NIR signal also appears to detect the expression observed at earlier times in this assay. As seen in FIG. 1, the μPET signal appears to be associated with a small region within the tissues, which colocalizes to the region of the injected cells. Further, tracking of the expression until the appearance of bone radiologically suggests that the region of MMP-9 expression within the tissues maps to the new bone. This is not surprising because the bone matrix has been known to be formed at the site of cartilage remodeling. What is intriguing is that the earlier nerve and vascular remodeling possibly detected by μPET is also mapping to the approximately same shape and size as the resultant bone formed during HO. The data indicates that HO is a localized event, and that intervention would perhaps be most selective if one were able to target the specific location. Furthermore, the results suggest that visualization of MMP-9 activity by μPET or NIR can provide an early indication of HO formation at the molecular level, and if a treatment were available, provide early diagnostics on its efficacy. Although there was an initial positive signal in the control region due to the inflammatory response launched by delivery of the adenovirus transduced cells, the inflammation and corresponding tracer uptake were transient. Tracer uptake related to HO, on the other hand, was evident within the tissues throughout the HO process.

The application of molecular imaging techniques to detect regions of soft tissues undergoing early HO in humans is a major advancement enabling early intervention. With the ability to better identify these regions, strategies that specifically target key molecules are implemented and lead to the development of effective therapies and conversely to stimulate bone regeneration in a controlled manner that involves the ECM.

### REFERENCES

All patents and publications cited herein are hereby incorporated by reference in their entirety herein. Full citations for the references cited herein are provided in the following list.

### PATENTS

U.S. Pat. No. 5,196,409
U.S. Pat. No. 7,378,395

### PUBLICATIONS

Achilefu, S., Jimenez, H. N., Dorshow, R. B., Bugaj, J. E., Webb, E. G., Wilhelm, R. R., Rajagopalan, R., Johler, J., and Erion, J. L. (2002) Synthesis, in vitro receptor binding, and in vivo evaluation of fluorescein and carbocyanine peptide-based optical contrast agents. J Med Chem 45, 2003-15.
Adams, K. E., Ke, S., Kwon, S., Liang, F., Fan, Z., Lu, Y., Hirschi, K., Mawad, M. E., Barry, M. A., and Sevick-Muraca, E. M. (2007) Comparison of visible and near-infrared wavelength-excitable fluorescent dyes for molecular imaging of cancer. J Biomed Opt 12, 024017.
Antunes, P., Ginj, M., Zhang, H., Waser, B., Baum, R. P., Reubi, J. C., and Maecke, H. (2007) Are radiogallium-labelled DOTA-conjugated somatostatin analogues superior to those labelled with other radiometals? Eur J Nucl Med Mol Imaging 34, 982-93.
Ardi V C, Kupriyanova T A, Deryugina E I, Quigley J P. Human neutrophils uniquely release TIMP-free MMP-9 to provide a potent catalytic stimulator of angiogenesis. Proc Natl Acad Sci USA. 104:20262-7. 2007.
Becker, A., Hessenius, C., Licha, K., Ebert, B., Sukowski, U., Semmler, W., Wiedenmann, B., and Grotzinger, C. (2001) Receptor-targeted optical imaging of tumors with near-infrared fluorescent ligands. Nat Biotechnol 19, 327-31.
Boschetto, P., Quintavalle, S., Zeni, E., Leprotti, S., Potena, A., Ballerin, L., Papi, A., Palladini, G., Luisetti, M., Annovazzi, L., Iadarola, P., De Rosa, E., Fabbri, L. M., and Mapp, C. E. (2006) Association between markers of emphysema and more severe chronic obstructive pulmonary disease. Thorax 61, 1037-42.
Breeman, W. A., de Jong, M., de Blois, E., Bernard, B. F., Konijnenberg, M., and Krenning, E. P. (2005) Radiolabelling DOTA-peptides with 68Ga. Eur J Nucl Med Mol Imaging 32, 478-85.
Cao, Q., Liu, S., Niu, G., Chen, K., Yan, Y., Liu, Z., and Chen, X. (2010) Phage display peptide probes for imaging early response to bevacizumab treatment. Amino Acids.
Chang K, Francis S A, Aikawa E, Figueiredo J L, Kohler R H, McCarthy J R, et al. Pioglitazone suppresses inflammation in vivo in murine carotid atherosclerosis: novel detection by dual-target fluorescence molecular imaging. Arterioscler Thromb Vasc Biol. 30:1933-9. 2010.
Chang, Y. H., Lin, I. L., Tsay, G. J., Yang, S. C., Yang, T. P., Ho, K. T., Hsu, T. C., and Shiau, M. Y. (2008) Elevated circulatory MMP-2 and MMP-9 levels and activities in patients with rheumatoid arthritis and systemic lupus erythematosus. Clin Biochem 41, 955-9.
Chen J, Tung C H, Allport J R, Chen S, Weissleder R, Huang P L. Near-infrared fluorescent imaging of matrix metalloproteinase activity after myocardial infarction. Circulation. 111:1800-5. 2005.
Chen, Y., Dhara, S., Banerjee, S. R., Byun, Y., Pullambhatla, M., Mease, R. C., and Pomper, M. G. (2009) A low molecular weight PSMA-based fluorescent imaging agent for cancer. Biochem Biophys Res Commun 390, 624-9.
Clever J L, Sakai Y, Wang R A, Schneider D B. Inefficient skeletal muscle repair in inhibitor of differentiation knockout mice suggests a crucial role for BMP signaling during adult muscle regeneration. Am J Physiol Cell Physiol. 298:C1087-99. 2010.
Cronin, C. G., Swords, R., Truong, M. T., Viswanathan, C., Rohren, E., Giles, F. J., O'Dwyer, M., and Bruzzi, J. F. (2010) Clinical utility of PET/CT in lymphoma. AJR Am J Roentgenol 194, W91-W103.
Deguchi J O, Aikawa M, Tung C H, Aikawa E, Kim D E, Ntziachristos V, et al. Inflammation in atherosclerosis: visualizing matrix metalloproteinase action in macrophages in vivo. Circulation. 114:55-62. 2006.
Deryugina, E. I., and Quigley, J. P. (2006) Matrix metalloproteinases and tumor metastasis. Cancer Metastasis Rev 25, 9-34.
Edwards, W. B., Xu, B., Akers, W., Cheney, P. P., Liang, K., Rogers, B. E., Anderson, C. J., and Achilefu, S. (2008) Agonist-antagonist dilemma in molecular imaging: evaluation of a monomolecular multimodal imaging agent for the somatostatin receptor. Bioconjug Chem 19, 192-200.
Fang K C, Raymond W W, Blount J L, Caughey G H. Dog mast cell alpha-chymase activates progelatinase B by cleaving the Phe88-Gln89 and Phe91-Glu92 bonds of the catalytic domain. J Biol Chem. 272:25628-35. 1997.
Forsberg J A, Pepek J M, Wagner S, Wilson K, Flint J, Andersen R C, et al. Heterotopic ossification in high-energy wartime extremity injuries: prevalence and risk factors. J Bone Joint Surg Am. 91:1084-91. 2009.
Fujimoto S, Hartung D, Ohshima S, Edwards D S, Zhou J, Yalamanchili P, et al. Molecular imaging of matrix metalloproteinase in atherosclerotic lesions: resolution with dietary modification and statin therapy. J Am Coll Cardiol. 52:1847-57. 2008.
Grams F, Brandstetter H, D'Alò S, Geppert D, Krell H W, Leinert H, Livi V, Menta E, Oliva A, Zimmermann G, et al. Pyrimidine-2,4,6-Triones: a new effective and selective class of matrix metalloproteinase inhibitors. Biol Chem. 2001; 382:1277-1285.
Gugala Z, Olmsted-Davis E A, Gannon F H, Lindsey R W, Davis A R. Osteoinduction by ex vivo adenovirus-mediated BMP2 delivery is independent of cell type. Gene Ther. 10:1289-96. 2003.
Hanaoka, H., Mukai, T., Habashita, S., Asano, D., Ogawa, K., Kuroda, Y., Akizawa, H., Lida, Y., Endo, K., Saga, T., and Saji, H. (2007) Chemical design of a radiolabeled gelatinase inhibitor peptide for the imaging of gelatinase activity in tumors. Nucl Med Biol 34, 503-10.
Hofmann, M., Maecke, H., Borner, R., Weckesser, E., Schoffski, P., Oei, L., Schumacher, J., Henze, M., Heppeler, A., Meyer, J., and Knapp, H. (2001) Biokinetics and imaging with the somatostatin receptor PET radioligand (68)Ga-DOTATOC: preliminary data. Eur J Nucl Med 28, 1751-7.
Houston J P, Ke S, Wang W, Li C, Sevick-Muraca E M. Quality analysis of in vivo nearinfrared fluorescence and conventional gamma images acquired using a dual-labeled tumortargeting probe. J Biomed Opt. 10:054010. 2005.
Hu J, Fiten P, Van den Steen P E, Chaltin P, Opdenakker G. Simulation of evolution-selected propeptide by high-throughput selection of a peptidomimetic inhibitor on a capillary DNA sequencer platform. Anal Chem. 2005; 77:2116-2124.
Kaijzel E L, van Heijningen P M, Wielopolski P A, Vermeij M, Koning G A, van Cappellen W A, et al. Multimodality imaging reveals a gradual increase in matrix metalloproteinase activity at aneurysmal lesions in live fibulin-4 mice. Circ Cardiovasc Imaging. 3:567-77. 2010.
Kimura, R. H., Miao, Z., Cheng, Z., Gambhir, S. S., and Cochran, J. R. (2010) A Dual-Labeled Knottin Peptide for PET and Near-Infrared Fluorescence Imaging of Integrin Expression in Living Subjects. Bioconjug Chem.
Koivunen, E., Arap, W., Valtanen, H., Rainisalo, A., Medina, O. P., Heikkila, P., Kantor, C., Gahmberg, C. G., Salo, T., Konttinen, Y. T., Sorsa, T., Ruoslahti, E., and Pasqualini, R. (1999) Tumor targeting with a selective gelatinase inhibitor. Nat Biotechnol 17, 768-74.
Kuhnast, B., Bodenstein, C., Haubner, R., Wester, H. J., Senekowitsch-Schmidtke, R., Schwaiger, M., and Weber, W. A. (2004) Targeting of gelatinase activity with a radiolabeled cyclic HWGF peptide. Nucl Med Biol 31, 337-44.
Kuil, J., Velders, A. H., and van Leeuwen, F. W. (2010) Multimodal tumortargeting peptides functionalized with both a radio- and a fluorescent label. Bioconjug Chem 21, 1709-19.
Lancelot, E., Amirbekian, V., Brigger, I., Raynaud, J. S., Ballet, S., David, C., Rousseaux, O., Le Greneur, S., Port, M., Lijnen, H. R., Bruneval, P., Michel, J. B., Ouimet, T., Rogues, B., Amirbekian, S., Hyafil, F., Vucic, E., Aguinaldo, J. G., Corot, C., and Fayad, Z. A. (2008) Evaluation of matrix metalloproteinases in atherosclerosis using a novel noninvasive imaging approach. Arterioscler Thromb Vasc Biol 28, 425-32.
Li, C., Wang, W., Wu, Q., Ke, S., Houston, J., Sevick-Muraca, E., Dong, L., Chow, D., Charnsangavej, C., and Gelovani, J. G. (2006) Dual optical and nuclear imaging in human melanoma xenografts using a single targeted imaging probe. Nucl Med Biol 33, 349-58.
Liu, Z., Liu, S., Niu, G., Wang, F., and Chen, X. (2010) Optical imaging of integrin alphavbeta3 expression with near-infrared fluorescent RGD dimer with tetra(ethylene glycol) linkers. Mol Imaging 9, 21-9.
Maecke, H. R., Hofmann, M., and Haberkorn, U. (2005) (68)Ga-labeled peptides in tumor imaging. J Nucl Med 46 Suppl I, 172S-8S.
Manduca, P., Castagnino, A., Lombardini, D., Marchisio, S., Soldano, S., Ulivi, V., Zanotti, S., Garbi, C., Ferrari, N., and Palmieri, D. (2009) Role of MT1-MMP in the osteogenic differentiation. Bone 44, 251-65.
Marshall, M. V., Rasmussen, J. C., Tan, I.-C. A., M. B., Adams, K. E., Wang, X., Fife, C. E., Maus, E. A., Smith, L. A., and Sevick-Muraca, E. M. (2010) Near-Infrared Fluorescence Imaging in Humans with Indocyanine Green: A Review and Update. The Open Surgical Oncology Journal 2, 12-25.
Meyer, G. J., Macke, H., Schuhmacher, J., Knapp, W. H., and Hofmann, M. (2004) 68Ga-labelled DOTA-derivatised peptide ligands. Eur J Nucl Med Mol Imaging 31, 1097-104.
Muroski, M. E., Roycik, M. D., Newcomer, R. G., Van den Steen, P. E., Opdenakker, G., Monroe, H. R., Sahab, Z. J., and Sang, Q. X. (2008) Matrix metalloproteinase-9/gelatinase B is a putative therapeutic target of chronic obstructive pulmonary disease and multiple sclerosis. Curr Pharm Biotechnol 9, 34-46.
Ogawa, M., Regino, C. A., Seidel, J., Green, M. V., Xi, W., Williams, M., Kosaka, N., Choyke, P. L., and Kobayashi, H. (2009) Dual-modality molecular imaging using antibodies labeled with activatable fluorescence and a radionuclide for specific and quantitative targeted cancer detection. Bioconjug Chem 20, 2177-84.
Ohshima S, Fujimoto S, Petrov A, Nakagami H, Haider N, Zhou J, et al. Effect of an antimicrobial agent on atherosclerotic plaques: assessment of metalloproteinase activity by molecular imaging. J Am Coll Cardiol. 55:1240-9. 2010.
Olmsted-Davis E, Gannon F H, Ozen M, Ittmann M M, Gugala Z, Hipp J A, et al. Hypoxic adipocytes pattern early heterotopic bone formation. Am J Pathol. 170:620-32. 2007.
Olmsted-Davis E A, Gugala Z, Gannon F H, Yotnda P, McAlhany R E, Lindsey R W, et al. Use of a chimeric adenovirus vector enhances BMP2 production and bone formation. Hum Gene Ther. 13:1337-47. 2002.
Ortega N, Behonick D J, Werb Z. Matrix remodeling during endochondral ossification. Trends Cell Biol. 14:86-93. 2004.
Paemen L, Martens E, Masure S, Opdenakker G. Monoclonal antibodies specific for natural human neutrophil gelatinase B used for affinity purification, quantitation by two-site ELISA and inhibition of enzymatic activity. Eur J Biochem. 1995; 234:759-765.
Poeppel, T. D., Krause, B. J., Heusner, T. A., Boy, C., Bockisch, A., and Antoch, G. (2009) PET/CT for the staging and follow-up of patients with malignancies. Eur J Radiol 70, 382-92.
Razavian M, Zhang J, Nie L, Tavakoli S, Razavian N, Dobrucki L W, et al. Molecular imaging of matrix metalloproteinase activation to predict murine aneurysm expansion in vivo. J Nucl Med. 51:1107-15. 2010.
Rodenberg, E., Azhdarinia, A., Lazard, Z., Hall, M., Kwon, S., Wilganowski, N., Merched-Sauvage, M., Salisbury, E. A., Davis, A. R., Sevick-Muraca, E. M., and Olmsted-Davis, E. (2010) MMP-9 as a biomarker of heterotopic ossification. Manuscript submitted for publication (copy on file with author).
Sampath, L., Kwon, S., Hall, M. A., Price, R. E., and Sevick-Muraca, E. M. (2010) Detection of Cancer Metastases with a Dual-labeled Near-Infrared/Positron Emission Tomography Imaging Agent. Transl Oncol 3, 307-217.
Sampath, L., Kwon, S., Ke, S., Wang, W., Schiff, R., Mawad, M. E., and Sevick-Muraca, E. M. (2007) Dual-labeled trastuzumab-based imaging agent for the detection of human epidermal growth factor receptor 2 overexpression in breast cancer. J Nucl Med 48, 1501-10.
Sampath, L., Wang, W., and Sevick-Muraca, E. M. (2008) Near infrared fluorescent optical imaging for nodal staging. J Biomed Opt 13, 041312.
Sevick-Muraca E M, Rasmussen J C. Molecular imaging with optics: primer and case for near-infrared fluorescence techniques in personalized medicine. J Biomed Opt. 13:041303. 2008.
Shafer J, Davis A R, Gannon F H, Fouletier-Dilling C M, Lazard Z, Moran K, et al. Oxygen tension directs chondrogenic differentiation of myelo-monocytic progenitors during endochondral bone formation. Tissue Engl. 3:2011-. 2007.
Shore E M, Kaplan F S. Inherited human diseases of heterotopic bone formation. Nat Rev Rheumatol. 6:518-27. 2010.
Sprague J E, Li W P, Liang K, Achilefu S, Anderson C J. In vitro and in vivo investigation of matrix metalloproteinase expression in metastatic tumor models. Nucl Med Biol. 33:227-37. 2006.
Sternlicht M D, Werb Z. How matrix metalloproteinases regulate cell behavior. Annu Rev Cell Dev Biol. 17:463-516. 2001.
Suzuki H, Sato M, Umezawa Y. Accurate targeting of activated macrophages based on synergistic activation of functional molecules uptake by scavenger receptor and matrix metalloproteinase. ACS Chem Biol. 3:471-9. 2008.
Tanaka, E., Choi, H. S., Humblet, V., Ohnishi, S., Laurence, R. G., and Frangioni, J. V. (2008) Real-time intraoperative assessment of the extrahepatic bile ducts in rats and pigs using invisible near-infrared fluorescent light. Surgery 144, 39-48.
Tchougounova E, Lundequist A, Fajardo I, Winberg J O, Abrink M, Pejler G. A key role for mast cell chymase in the activation of pro-matrix metalloprotease-9 and pro-matrix metalloprotease-2. J Biol Chem. 280:9291-6. 2005.
Jennifer Vandooren, Nathalie Geurts, Erik Martens, Philippe E Van den Steen, Steven De Jonghe, Piet Herdewijn, and Ghislain Opdenakker. Gelatin degradation assay reveals MMP-9 inhibitors and function of O-glycosylated domain. World J Biol Chem. 2011 January 26; 2(1): 14-24.
Velikyan, I., Beyer, G. J., and Langstrom, B. (2004) Microwave-supported preparation of (68)Ga bioconjugates with high specific radioactivity. Bioconjug Chem 15, 554-60.
Virgolini, I., Ambrosini, V., Bomanji, J. B., Baum, R. P., Fanti, S., Gabriel, M., Papathanasiou, N. D., Pepe, G., Oyen, W., De Cristoforo, C., and Chiti, A. (2010) Procedure guidelines for PET/CT tumour imaging with 68Ga-DOTA-conjugated peptides: 68Ga-DOTA-TOC, 68Ga-DOTA-NOC, 68Ga-DOTA-TATE. Eur J Nucl Med Mol Imaging 37, 2004-10.
Wang C, Sevick-Muraca E M. Dual-labeled targeting imaging agents for hybrid optical and nuclear molecular imaging. In: Chen X, ed. Recent Advances of Bioconjugation Chemistry in Molecular Imaging: Signpost Books; 2009.
Wang WaEMS-M. Dual-labeled, targeting imaging agents for hybrid optical and nuclear molecular imaging. In: Chen X, ed. Molecular Imaging: Research Signpost; 2010
Wang Y, Imitola J, Rasmussen S, O'Connor K C, Khoury S J. Paradoxical dysregulation of the neural stem cell pathway sonic hedgehog-Gli1 in autoimmune encephalomyelitis and multiple sclerosis. Ann Neurol. 64:417-27. 2008.
Wang, W., Ke, S., Wu, Q., Charnsangavej, C., Gurfinkel, M., Gelovani, J. G., Abbruzzese, J. L., Sevick-Muraca, E. M., and Li, C. (2004) Near-infrared optical imaging of integrin alphavbeta3 in human tumor xenografts. Mol Imaging 3, 343-51.
Wang, W., Shao, R., Wu, Q., Ke, S., McMurray, J., Lang, F. F., Jr., Charnsangavej, C., Gelovani, J. G., and Li, C. (2009) Targeting gelatinases with a near-infrared fluorescent cyclic His-Try-Gly-Phe peptide. Mol Imaging Biol 11, 424-33.
Williams, A. T. R., Winfield, S. A., and J. N., M. (1983) Relative fluorescence quantum yields using a computer controlled luminescence spectrometer. Analyst 108, 1067-1071
Yao Y, Nowak S, Yochelis A, Garfinkel A, Bostrom K I. Matrix G L A protein, an inhibitory morphogen in pulmonary vascular development. J Biol Chem.282:30131-42. 2007.
Zhernosekov, K. P., Filosofov, D. V., Baum, R. P., Aschoff, P., Bihl, H., Razbash, A. A., Jahn, M., Jennewein, M., and Rosch, F. (2007) Processing of generator-produced 68Ga for medical application. J Nucl Med 48, 1741-8.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of identifying a site of heterotopic bone formation in an individual or identifying a site at risk of developing heterotopic bone formation in an individual, comprising the step of providing to a localized site in the individual a MMP-9 and/or MMP-2 targeting agent, said agent comprising a first and a second imaging moiety.

2. The method of claim 1, wherein the localized site is muscle, a vessel, a joint, aortic valve, tendons, or ligaments.

3. The method of claim 1, wherein the localized site is selected from the group consisting of:
a) a trauma site;
b) a site subjected to repetitive motion;
c) a site at risk for aneurysm; and
d) joint arthoplasty.

4. The method of claim 1, wherein the individual has or is at risk of having traumatic brain injury, atherosclerosis, aortic stenosis, calcific aortic valve disease, spinal cord injury, joint replacement, or amputation.

5. The method of claim 1, wherein the individual has one or more of the risk factors selected from the group consisting of male gender; has active ankylosing spondylitis; has diffuse Idiopathic Skeletal Hyperostosis; has post traumatic arthritis; has heterotrophic osteoarthritis; has previous heterotopic ossification; had previous hip fusion; has Paget's disease; has Parkinson's disease has excessive osteophytosis or enthesiopathic radiographic changes on AP of pelvis; has traumatic brain injury and/or spinal cord injury and/or stroke; has had hip surgery or other joint surgery; has burns; has long period of immobility; has a joint infection; has trauma to muscle or soft tissue; and a combination thereof.

6. The method of claim 1, wherein the agent is a peptide, small molecular, polypeptide, antibody, nucleic acid, or combination thereof.

7. The method of claim 1, wherein the first and second imaging moieties are selected from the group consisting of an optical imaging moiety, a fluorescent imaging moiety, and a nuclear imaging moiety.

8. The method of claim 1, wherein a first imaging moiety is a radionuclide and a second imaging moiety is a NIR fluorophore.

9. The method of claim 1, wherein the agent is delivered locally.

10. The method of claim 1, wherein the agent is delivered by injection.

11. The method of claim 1, further comprising the step of providing to the individual a therapeutically effective amount of one or more agents that treat heterotopic bone growth.

12. The method of claim 11, wherein the MMP-9 and/or MMP-2 targeting agent is the agent that treats heterotopic bone growth.

13. The method of claim 11, wherein the one or more agents that treat heterotopic bone growth is physical therapy, bisphosphonate drug; nonsteroidal anti-inflammatory drugs (NSAIDs); radiation therapy; and/or surgery, TRVP1 antagonists, RAR alpha antagonists, substance P antagonists, MMP9 inhibitors, or a combination thereof.
